(19) United States
(12) Patent Application Publication
(10) Pub. No.: US 2017/0135933 A1
(43) Pub. Date: May 18, 2017

(54) PHOTOPROTECTIVE COMPOSITIONS COMPRISING PHOTOSENSITIVE 1,3,5-TRIAZINE COMPOUNDS, DIBENZOYLMETHANE COMPOUNDS AND SILICEOUS S-TRIAZINES SUBSTITUTED WITH TWO AMINOBENZOATE OR AMINOBENZAMIDE GROUPS

(71) Applicant: L'OREAL, Paris (FR)

(72) Inventor: Didier CANDAU, Bievres (FR)

(73) Assignee: L'OREAL, Paris (FR)

(21) Appl. No.: 15/237,939

(22) Filed: Aug. 16, 2016

Related U.S. Application Data
(63) Continuation of application No. 11/987,212, filed on Nov. 28, 2007, now abandoned.
(60) Provisional application No. 60/873,000, filed on Dec. 6, 2006.

(30) Foreign Application Priority Data
Nov. 28, 2006 (FR) 0655164

(51) Int. Cl. A61K 8/58; A61Q 19/04; A61Q 17/04; A61K 8/891; A61K 8/49; A61K 8/35

(52) U.S. Cl. CPC A61K 8/585; A61K 8/4966; A61K 8/35; A61Q 17/04; A61K 8/891; A61Q 19/04; A61K 2800/52

(57) ABSTRACT

UV-photoprotective, topically applicable cosmetic/dermatological compositions contain:
(a) at least one dibenzoylmethane compound,
(b) at least one 1,3,5-triazine compound that is photosensitive in the presence of a dibenzoylmethane compound, and
(c) at least one siliceous s-triazine compound substituted with two aminobenzoate or aminobenzamide groups, or a tautomeric form thereof, the 1,3,5-triazine compounds being improvedly photostable in such compositions.

### CROSS-REFERENCE TO PRIORITY/PROVISIONAL APPLICATIONS

This application claims priority under 35 U.S.C. §119 of FR 06/55164, filed Nov. 28, 2006, and of U.S. Provisional Application No. 60/873,000, Dec. 6, 2006, each hereby expressly incorporated by reference and each assigned to the assignee hereof.

### BACKGROUND OF THE INVENTION

### Technical Field of the Invention

The present invention relates to compositions comprising, formulated into a physiologically acceptable support, at least one screening system which comprises:
(a) at least one UV-screening agent of the dibenzoylmethane derivative type, and
(b) at least one UV-screening agent of the 1,3,5-triazine type that is photosensitive in the presence of a dibenzoylmethane derivative, and
(c) at least one siliceous s-triazine compound substituted with two aminobenzoate or aminobenzamide groups of formula (I), or a tautomeric form thereof, more fully described below.

Description of Background and/or Related and/or Prior Art

It is known that light radiation with wavelengths of from 280 nm and 400 nm promote tanning of the human epidermis and that light rays with wavelengths more particularly from 280 and 320 nm, known as UV-B rays, cause skin burns and erythema which can harm the development of a natural tan. For these reasons, as well as for aesthetic reasons, there is a constant demand for means of controlling this natural tanning in order thus to control the color of the skin; this UV-B radiation should thus be screened out.

It is also known that UV-A rays, with wavelengths from 320 and 400 nm, which cause browning of the skin, are liable to induce adverse changes therein, in particular in the case of sensitive skin or skin which is continually exposed to solar radiation. UV-A rays cause in particular a loss of elasticity of the skin and the appearance of wrinkles leading to premature aging of the skin. These promote triggering of the erythemal reaction or amplify this reaction in certain individuals and may even be the cause of phototoxic or photoallergic reactions. Thus, for aesthetic and cosmetic reasons such as the conservation of the skin's natural elasticity, for example, an increasingly large number of individuals wish to control the effect of UV-A rays on their skin. It is thus desirable also to screen out UV-A radiation.

In this respect, one particularly advantageous family of UV-A screening agents currently consists of dibenzoylmethane derivatives, and in particular 4-tert-butyl-4′-methoxydibenzoylmethane, which have high intrinsic absorbing power. These dibenzoylmethane derivatives, which are now products that are well known per se as screening agents that are active in the UV-A range, are described in particular in FR-A-2,326,405 and FR-2,440,933, as well as in EP-A-0,114,607; 4-tert-butyl-4′-methoxydibenzoylmethane is moreover currently marketed under the trademark Parsol 1789 by Hoffmann LaRoche.

1,3,5-Triazine derivatives are particularly desired in anti-sun/sunscreen cosmetics due to the fact that they are highly active in the UV-B range, and even in the UV-A range for certain of these compounds, depending on the nature of the substituents involved. They are especially described in U.S. Pat. No. 4,367,390, EP-863,145, EP-517,104, EP-570,838, EP-507,691, EP-796,851, EP-775,698, EP-878,469 and EP-933,376, and the following are known in particular:

2,4,6-tris[p-(2′-ethylhexyl-1′-oxycarbonyl)anilino]-1,3,5-triazine or Ethylhexyl Triazone (INCI name), marketed under the trademark Uvinul T 150 by BASF,

2-[(p-(tert-butylamido)anilino]-4,6-bis[(p-(2′-ethylhexyl-1′-oxycarbonyl)anilino]-1,3,5-triazine or Diethylhexyl Butamido Triazone (INCI name), marketed under the trademark Uvasorb HEB by Sigma 3V. They have high UV-B absorbing power and it would therefore be very advantageous to be able to use them in combination with 4-tert-butyl-4′-methoxydibenzoylmethane mentioned above for the purpose of obtaining products that afford broad and effective protection against all UV radiation.

However, it has been found that certain of these 1,3,5-triazine derivatives are photosensitive when they are in the presence of 4-tert-butyl-4′-methoxydibenzoylmethane, i.e. under UV irradiation, they have the drawback of undergoing substantial chemical degradation. Under these conditions, the combination of the two screening agents no longer affords prolonged, broad anti-sun protection to the skin and the hair.

### SUMMARY OF THE INVENTION

It has now surprisingly been determined that the introduction of a siliceous s-triazine substituted with two aminobenzoate or aminobenzamide groups, notably of particular formula (I), in a composition containing a dibenzoylmethane derivative, in particular 4-tert-butyl-4′-methoxydibenzoylmethane, in combination with at least one photosensitive 1,3,5-triazine derivative, and in particular with 2,4,6-tris[p-(2′-ethylhexyl-1′-oxycarbonyl)anilino]-1,3,5-triazine, makes it possible, quite remarkably, to improve the photostability of this 1,3,5-triazine derivative in such compositions, and thus the overall efficacy of these compositions.

This discovery constitutes the basis of the present invention.

The present invention thus features compositions comprising, formulated into a physiologically acceptable support, at least one screening system, which comprises:

(a) at least one UV-screening agent of the dibenzoylmethane derivative type, and

(b) at least one UV-screening agent of the 1,3,5-triazine type that is photosensitive in the presence of a dibenzoylmethane derivative, and

(c) at least one siliceous s-triazine compound substituted with two aminobenzoate or aminobenzamide groups of formula (I), or a tautomeric form thereof, which will be described in greater detail hereinbelow.

Thus, according to the present invention, cosmetic or dermatological compositions may be produced containing 4-tert-butyl-4′-methoxydibenzoylmethane in combination with at least one photosensitive 1,3,5-triazine derivative, in which compositions the concentration of 1,3,5-triazine derivative remains relatively constant even if these compositions are subjected to the action of light.

The present invention also features a process for improving the stability to UV radiation (photostability) of a 1,3,5-triazine derivative that is photosensitive in the presence of a dibenzoylmethane derivative, comprising adding to the said combination at least one siliceous s-triazine compound substituted with two aminobenzoate or aminobenzamide groups of formula (I), or a tautomeric form thereof, which will be described in greater detail hereinbelow.

This invention also features the use of at least one siliceous s-triazine compound substituted with two aminobenzoate or aminobenzamide groups of formula (I), or a tautomeric form thereof, in a composition containing at least one dibenzoylmethane derivative and at least one 1,3,5-triazine derivative that is photosensitive in the presence thereof, for the purpose of improving the photostability of the said 1,3,5-triazine derivative.

Other characteristics, aspects and advantages of the present invention will become apparent from the detailed description that follows.

Herein, the expression “system for screening out UV radiation” means an agent for screening out UV radiation constituted either of a single organic or mineral compound for screening out UV radiation, or of a mixture of several organic or mineral compounds for screening out UV radiation, for example a mixture comprising a UV-A screening agent and a UV-B screening agent.

The term “cosmetically acceptable” means compatible with the skin and/or its integuments, which has a pleasant color, odor and feel and which does not give rise to any unacceptable discomfort (stinging, tautness or redness) liable to put the consumer off using this composition.

The expression “screening agent that is photosensitive in the presence of a dibenzoylmethane derivative” means any screening agent capable of chemically degrading under UV irradiation in the presence of a dibenzoylmethane derivative; which is reflected by a decrease in the amount of screening agent in the composition and/or a loss of anti-sun efficacy after irradiation.

According to the present invention, the siliceous s-triazine compounds of formula (I) are used in an amount that is sufficient to obtain an appreciable and significant improvement in the photostability of the 1,3,5-triazine derivative in a given composition containing a dibenzoylmethane derivative. This minimum amount of photostabilizer may vary according to the amount of triazine and of dibenzoylmethane present at the start in the composition and according to the nature of the cosmetically acceptable support selected for the composition. It may be determined without any difficulty by means of a standard test for measuring photostability.

### DETAILED DESCRIPTION OF BEST MODE AND SPECIFIC/PREFERRED EMBODIMENTS OF THE INVENTION

Among the dibenzoylmethane derivatives in accordance with the invention, especially preferred are;
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane,
2,6-dimethyl-4-tert-butyl-4′-methoxydibenzoylmethane.

Among the dibenzoylmethane derivatives indicated above, particularly preferred is 4-isopropyldibenzoylmethane, marketed under the trademark Eusolex 8020 by Merck, and corresponding to the following formula:

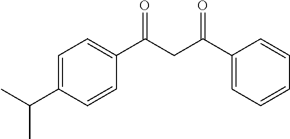 [SMILES: CC(C)c1ccc(C(=O)CC(=O)c2ccccc2)cc1]

It is most particularly preferred to employ 4-(tert-butyl)-4′-methoxydibenzoylmethane or Butylmethoxydibenzoylmethane, which is marketed under the trademark Parsol 1789 by Roche Vitamins: this screening agent corresponds to the following formula:

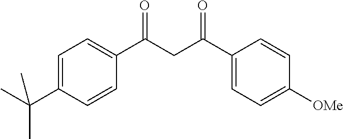 [SMILES: COc1ccc(C(=O)CC(=O)c2ccc(C(C)(C)C)cc2)cc1]

The dibenzoylmethane derivative(s) may be present in the compositions in accordance with the invention in contents preferably ranging from 0,01% to 20% by weight, more preferentially from 0.1% to 10% by weight and even more preferentially from 0,1% to 6% by weight relative to the total weight of the composition.

The siliceous s-triazine compounds substituted with two aminobenzoate or aminobenzamide groups in accordance with the invention correspond to the general formula (I) below, or a tautomeric form thereof:

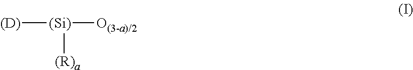

in which:

the radicals R, which may be identical or different, are each a linear or branched and optionally halogenated or unsaturated C₁-C₃₀ alkyl radical, a C₆-C₁₂ aryl radical, a C₁-C₁₀ alkoxy radical or a trimethylsilyloxy group;

a=0 to 3;

the group D is an s-triazine compound of formula (II) below:

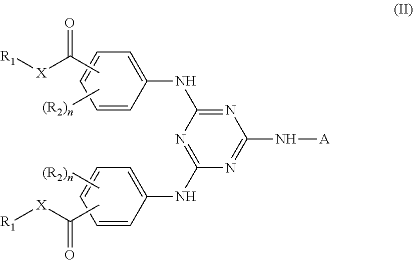

in which:

X is —O— or —NR₃—, with R₃ representing hydrogen or a C₁-C₅ alkyl radical,

R₁ is a linear or branched and optionally unsaturated C₁-C₂₀ alkyl radical optionally containing a silicon atom, a C₅-C₂₀ cycloalkyl group optionally substituted with 1 to 3 linear or branched C₁-C₄ alkyl radicals, the group —(CH₂CHR₄—O)ₘR₅ or the group —CH₂—CH(OH)—CH₃—O—R₈,

R₄ is hydrogen or methyl; the group (C═O)XR₁, which may be in an ortho, meta or para position relative to the amino group,

R₅ is hydrogen or a C₁-C₈ alkyl group,

R₆ is hydrogen or a C₄-C₈ alkyl group,

m is an integer ranging from 2 to 20,

n=0 to 2,

the radicals R₂, which may be identical or different, are each a hydroxyl radical, a linear or branched C₁-C₈ alkyl radical or a C₁-C₈ alkoxy radical, two adjacent groups R₂ on the same aromatic nucleus may together form an alkylidenedioxy group in which the alkylidene group contains 1 or 2 carbon atoms,

A is a divalent radical selected from among methylene, —[CHSi(CH₃)₃]—, ethylene and a group corresponding to one of the formulae (Ill), (IV) and (V) below:

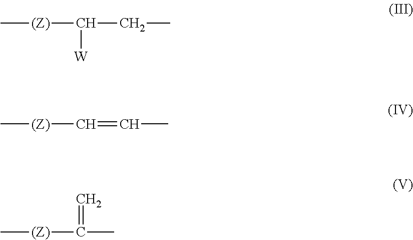

in which:

Z is a linear or branched, saturated or unsaturated C₁-C₁₀ alkylene diradical optionally substituted with a hydroxyl radical or oxygen and optionally containing an amino group,

W is a hydrogen atom, a hydroxyl radical or a linear or branched, saturated or unsaturated C₁-C₈ alkyl radical.

It should be noted that the derivatives of formula (I) may be used in their tautomeric forms and more particularly in the tautomeric form of formula (I′) below:

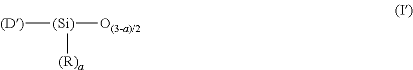

in which the group D′ is an s-triazine compound of formula (II′) below:

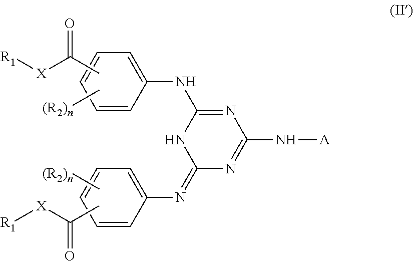

In addition to the units of formula -A-(Si)(R)ₐ(O)_{(3-a)/2}, the organosiloxane may comprise units of formula (R)_{b}—(Si)(O)_{(4-b)/2} in which:

R has the same definition as in formula (I),

b=1, 2 or 3.

The preferred s-triazine derivatives are those for which, in formula (II) or (II′), at least one, and even more preferentially all, of the following characteristics are satisfied:

R is methyl,

a=1 or 2,

X is O,

R₁ is a C₄—O₅ radical,

n=0,

the group (C═O)XR₁ is in the para position relative to the amino group,

Z═—CH₂—,

W═H.

Preferably, the s-triazine compounds of the invention are represented by formula (Ia), (Ib) or (lc) below:

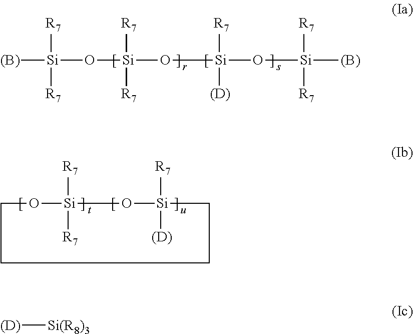

in which:

(D) corresponds to formula (II) as defined above,

the radicals R₇, which may be identical or different, are each a linear or branched C₁-C₂₀ alkyl, phenyl, 3,3,3-trifluoropropyl or trimethylsilyloxy radical or a hydroxyl radical,

the radicals R₈, which may be identical or different, are each a linear or branched C₁-C₂₀ alkyl or alkenyl radical, or hydroxyl or phenyl radical,

the radicals (B), which may be identical or different, are each a radical R₇ or a radical (D),

r is an integer ranging from 0 to 200 inclusive,

s is an integer ranging from 0 to 50 and if s=0, at least one of the two symbols (B) is (D),

u is an integer ranging from 1 to 10,

t is an integer ranging from 0 to 10, with the proviso that t+u is greater than or equal to 3, and also the tautomeric forms thereof.

The linear diorganosiloxanes of formula (Ia) are particularly preferred.

The linear or cyclic diorganosiloxanes of formula (Ia) or (Ib) according to the present invention are random oligomers or polymers preferably having at least one, and even more preferentially all, of the following characteristics:

R₇ is a methyl radical or a hydroxyl radical,

B is preferentially methyl (which is the case for the linear compounds of formula (Ia)),

As examples of compounds of formula (I) that are particularly preferred, representative are the compounds of formulae (a) to (m) below, and also the tautomeric forms thereof:

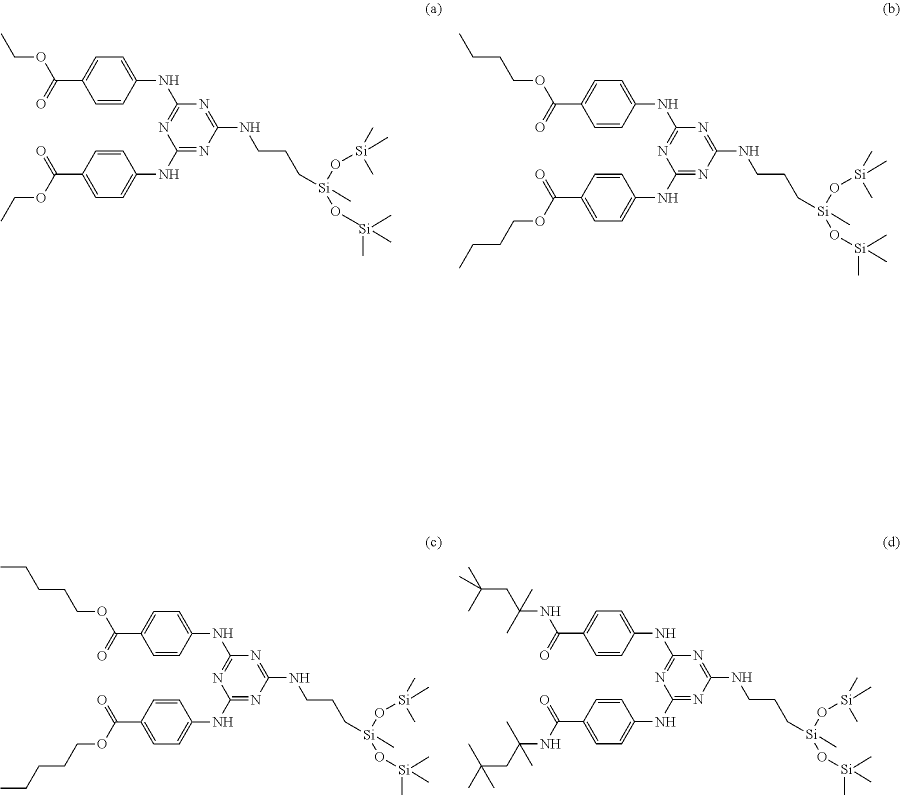

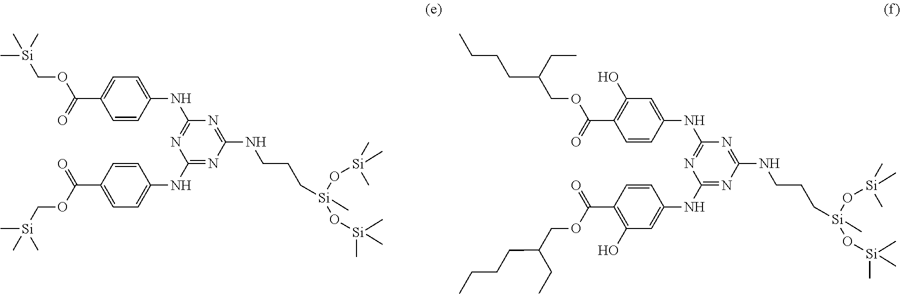

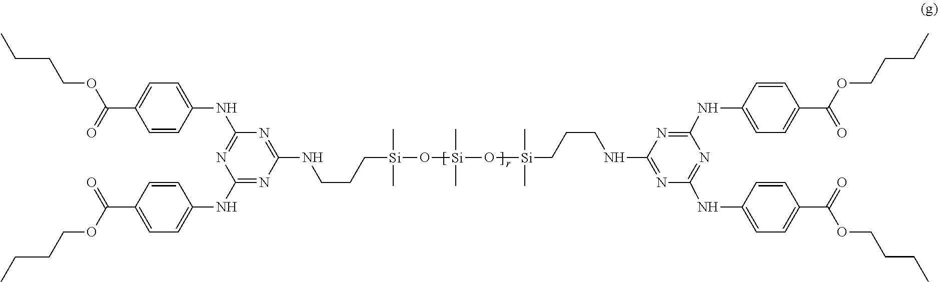

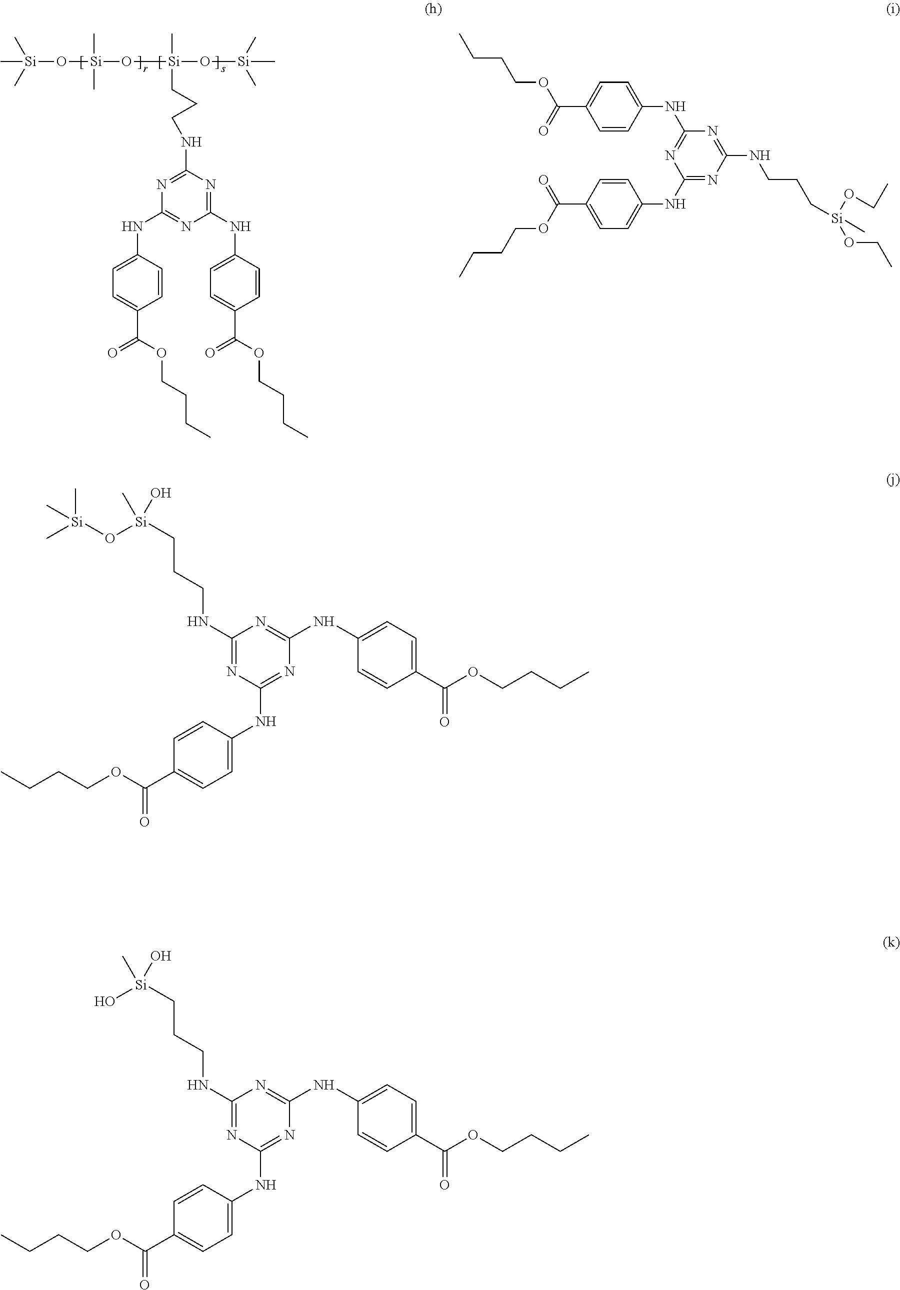

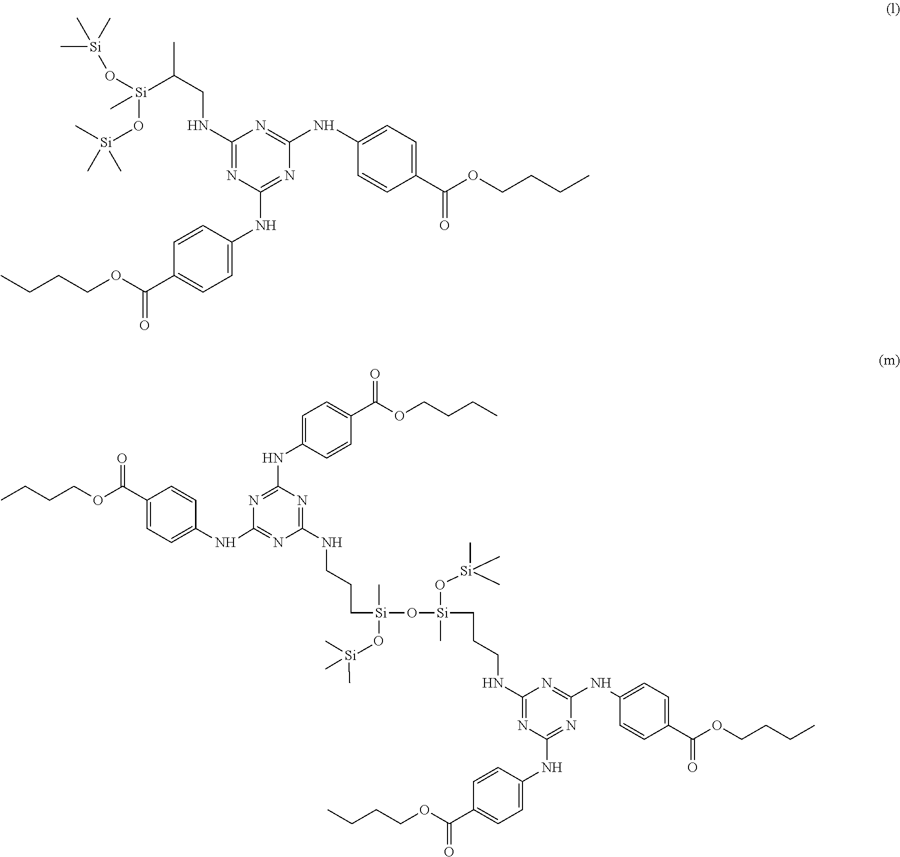

More particularly preferred is the compound 2,4-bis(n-butyl 4′-diylaminobenzoate)-6-{[1,3,3,3-tetramethyl-1-[(trimethylsilyl)oxy]disiloxanyl]propyl-3-ylamino}-s-triazine of structure (b):

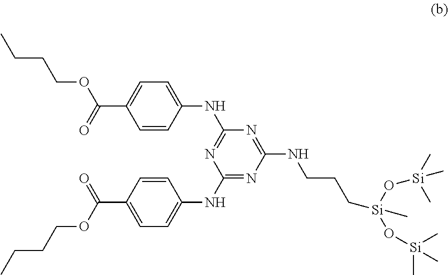 [SMILES: CCCCOC(=O)c1ccc(Nc2nc(NCCC[Si](C)(O[Si](C)(C)C)O[Si](C)(C)C)nc(Nc3ccc(C(=O)OCCCC)cc3)n2)cc1]

The compounds of formula (I) may be prepared according to the reaction scheme below;

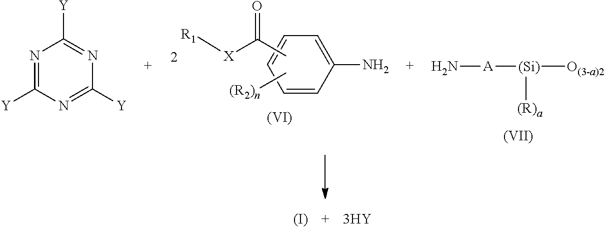

in which R, R₁, R₂, A, n and a correspond to the above definitions and Y is a halogen, in particular chlorine or bromine.

The order of introduction of the reagents may be performed, impartially, as follows; 2 equivalents of the derivative of formula (VI) followed by 1 equivalent of the derivative of formula (VII) (route 1), or 1 equivalent of the derivative of formula (VII) followed by 2 equivalents of the derivative of formula (VI) (route 2),

The above reactions may optionally be performed in the presence of a solvent (for example: THF, acetone/water for the first step; toluene, xylene or 1,2-dichloroethane for the second step), at a temperature of from 0° C. to 200° C. and more particularly from 0° C. to 20° C. for the first step, and from 50° to 120° C. for the second step, and in the presence or absence of a base for scavenging the acid formed (for example: sodium bicarbonate, sodium carbonate, aqueous sodium hydroxide, triethylamine or pyridine). They may also be performed in microwave reactors in the presence or absence of a solvent (for example: toluene, xylene or 1,2-dichloroethane) or in the presence or absence of 10% graphite, at a temperature of from 50° to 150° C., at a power of 50-150 watts for a period of 10 to 30 minutes.

When a is equal to 1-3 and R is an alkoxy, the alkoxysilane monomer derivatives may be polymerized via standard methods of silicone chemistry.

The preparation of the benzoic acid amine derivatives of formula (VI) is described especially in FR-2,151,503. As benzoic acid amine derivatives that are particularly suitable for preparing the compounds according to the invention, butyl 4-aminobenzoate and pentyl 4-aminobenzoate are exemplary.

The amino silicones of formula (VII) may be obtained from Dow Corning Toray Silicone Co. Ltd, such as those of α,ω-diamino structure, for instance BY16-853 (viscosity: 30; NH₂ equivalent: 650) or BY16-853B (viscosity: 80; NH₂ equivalent: 2200) or those of side-group structure, for instance BY16-828 (viscosity: 120; NH₂ equivalent: 3500) or BY16-850 (viscosity: 1100; NH₂ equivalent: 4000).

An exemplary aminomethyltrimethylsilane is marketed by Gelest and is bis(trimethylsilyl)methylamine (RN 134340-00-4).

The siliceous s-triazine compounds of formula (I) in accordance with the invention are preferably present in the compositions in accordance with the invention in contents ranging from 0.01% to 20% by weight, more preferentially from 0.1% to 10% to even more preferentially from 0.1% to 6% by weight relative to the total weight of the composition.

The UV-screening agents of the photosensitive 1,3,5-triazine type in accordance with the invention are preferably selected from among the 1,3,5-triazine derivatives of formula (VIII) Belo

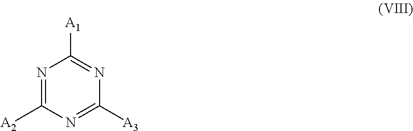

in which the radicals A₁, A₂ and A₃, which may be identical or different, are selected from among the groups of formula (IX):

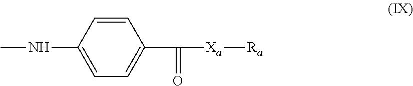

in which:

Xₐ, which may be identical or different, represent oxygen or the radical —NH—;

the radicals Rₐ, which may be identical or different; are selected from among hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a polyoxyethylene radical comprising from 1 to 6 ethylene oxide units, the terminal OH group of which is methylated; a radical of formula (X), (XI) or (XII) below:

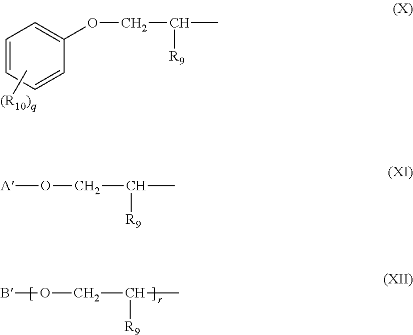

in which:

R₉ is hydrogen or a methyl radical;

R₁₀ is a C₁-C₉ alkyl radical;

q is an integer ranging from 0 to 3;

r is an integer ranging from 1 to 10;

A′ is a C₄-C₈ alkyl radical or a C₅-C₈ cycloalkyl radical;

B′ is selected from among a linear or branched C₁-C₈ alkyl radical; a C₅-C₈ cycloalkyl radical; an aryl radical optionally substituted with one or more C₁-C₄ alkyl radicals.

A first family of 1,3,5-triazine derivatives that is more particularly preferred, and that is described especially in EP-A-0,517,104, is that of the 1,3,5-triazines corresponding to formula (VIII) in which A₁, A₂ and A₃ are of formula (IX) and have the following characteristics:

one of the radicals Xₐ—Rₐ is a radical —NH—Rₐ with Rₐ selected from a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical;

the other two radicals Xₐ—Rₐ are each a radical —O—R, with Rₐ, which may be identical or different, selected from among hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical.

A second family of 1,3,5-triazine derivatives that is more particularly preferred, and that is described especially in EP-A-0,570,838, is that of the 1,3,5-triazines corresponding to formula (VIII) in which A₁, A₂ and A₃ are of formula (IX) and have all of the following characteristics:

one or two groups Xₐ—Rₐ are each a radical —NH—Rₐ, with Rₐ selected from a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical;

the other or the other two group(s) Xₐ—Rₐ being a radical —O—Rₐ with Rₐ, which may be identical or different, selected from among hydrogen; an alkali metal; an ammonium radical optionally substituted with one or more alkyl or hydroxyalkyl radicals; a linear or branched C₁-Cₐ alkyl radical; a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical.

A 1,3,5-triazine of this second family that is particularly preferred is 2-[(p-(tert-butylamido)anilino]-4,6-bis[(p-(2′-ethylhexyl-1′-oxycarbonyl)anilino]-1,3,5-triazine or Diethylhexyl Butamido Triazone marketed under the trademark Uvasorb HEB by Sigma 3V and corresponding to the following formula:

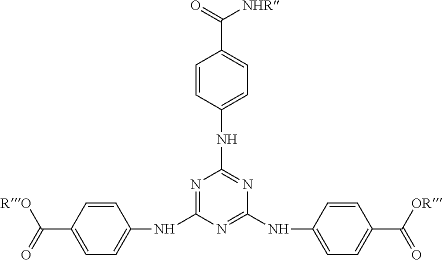

in which R′″ is a 2-ethylhexyl radical and R″ is a tert-butyl radical.

A third preferred family of compounds that may be employed according to the context of the present invention, and which is described especially in U.S. Pat. No. 4,724,137, is that of the 1,3,5-triazines corresponding to formula (VIII) in which A₁, A₂ and A₃ are of formula (IX) and have the following characteristics:

Xₐ are identical and represent oxygen;

the radicals Rₐ, which may be identical or different, are each a C₆-C₁₂ alkyl radical or a polyoxyethylene radical comprising from 1 to 6 ethylene oxide units and in which the terminal OH group is methylated.

A 1,3,5-triazine of this third family that is particularly preferred is 2,4,6-tris[p(2′-ethylhexyl-1′-oxycarbonyl)anilino]-1,3,5-triazine or Ethylhexyl Triazone marketed especially under the trademark Uvinul T 150 by BASF and corresponding to the following formula:

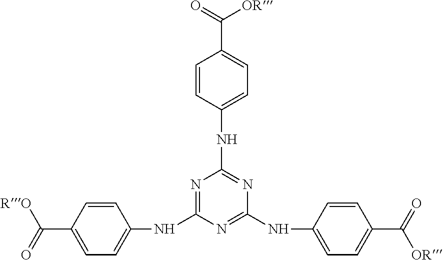

in which R′″ is a 2-ethylhexyl radical.

The photosensitive 1,3,5-triazine compounds are preferably present in the compositions in accordance with the invention in contents ranging from 0.01% to 20% by weight, more preferentially from 0.1% to 10% to even more preferentially from 0.1% to 6% by weight relative to the total weight of the composition.

The compositions according to the invention are generally suitable for topical application to the skin and thus generally comprise a physiologically acceptable medium, i.e., a medium that is compatible with the skin and/or its integuments (hair, eyelashes, eyebrows and nails). It is preferably a cosmetically acceptable medium, i.e. a medium that has a pleasant color, odor and feel and that does not cause any unacceptable discomfort (stinging, tautness or redness) liable to dissuade the consumer from using this composition.

The compositions in accordance with the invention may also comprise other additional UV-A active and/or UV-B active organic or mineral UV-screening agents.

Needless to say, one skilled in the art will take care to select the optional additional screening agent(s) and/or the amounts thereof such that the advantageous properties intrinsically associated with the compositions in accordance with the invention are not, or are not substantially, adversely affected by the envisaged addition(s).

The additional organic photoprotective agents are selected especially from among anthranilates; cinnamic derivatives; salicylic derivatives; camphor derivatives; benzophenone derivatives; β,β-diphenylacrylate derivatives; benzalmalonate derivatives; benzimidazole derivatives; imidazolines; bis-benzazolyl derivatives as described in EP-669,323 and U.S. Pat. No. 2,463,264; p-aminobenzoic acid (PABA) derivatives; benzoxazole derivatives as described in EP-0,832,642, EP-1,027,883, EP-1,300,137 and DE-10162844; screening polymers and screening silicones such as those described especially in WO 93/04665; α-alkylstyrene-based dimers, such as those described in DE-19855649; 4,4-diarylbutadienes such as those described in EP-0,967,200, DE-19746654, DE-19755649, EP-A-1,008,586, EP-1,133,980 and EP-133,981, and mixtures thereof.

As examples of additional organic photoprotective agents, representative are those denoted hereinbelow under their INCI name:

Para-Aminobenzoic Acid Derivatives:

### PABA,

### Ethyl PABA,

Ethyl dihydroxypropyl PABA,
Ethylhexyl dimethyl PABA marketed in particular under the trademark Escalol 507 by ISP,

### Glyceryl PABA,

PEG-25 PABA marketed under the trademark Uvinul P25 by BASF,

Salicylic Derivatives:

Homosalate marketed under the trademark Eusolex HMS by Rona/EM Industries,
Ethylhexyl salicylate marketed under the trademark Neo Heliopan OS by Haarmann and Reimer,
Dipropylene glycol salicylate marketed under the trademark Dipsal by Scher, TEA salicylate marketed under the trademark Neo Heliopan TS by Haarmann and Reimer,

Cinnamic Derivatives:

Ethylhexyl methoxycinnamate marketed in particular under the trademark Parsol MCX by Hoffmann LaRoche,
Isopropyl methoxycinnamate,
Isoamyl methoxycinnamate marketed under the trademark Neo Heliopan E 1000 by Haarmann and Reimer,

### Cinoxate,

DEA methoxycinnamate,
Diisopropyl methylcinnamate,
Glyceryl ethylhexanoate dimethoxycinnamate,

β,β-Diphenylacrylate Derivatives:

Octocrylene marketed in particular under the trademark Uvinul N539 by BASF,
Etocrylene marketed in particular under the trademark Uvinul N35 by BASF,

Benzophenone Derivatives:

Benzophenone-1 marketed under the trademark Uvinul 400 by BASF,
Benzophenone-2 marketed under the trademark Uvinul D50 by BASF,
Benzophenone-3 or Oxybenzone marketed under the trademark Uvinul M40 by BASF,
Benzophenone-4 marketed under the trademark Uvinul MS40 by BASF, Benzophenone-5,
Benzophenone-6 marketed under the trademark Helisorb 11 by Norquay,
Benzophenone-8 marketed under the trademark Spectra-Sorb UV-24 by American Cyanamid,
Benzophenone-9 marketed under the trademark Uvinul DS-49 by BASF, Benzophenone-12,

Benzylidenecamphor Derivatives:

3-Benzylidenecamphor manufactured under the trademark Mexoryl SD by Chimex,
4-Methylbenzylidenecamphor marketed under the trademark Eusolex 6300 by Merck,
Benzylidenecamphorsulfonic acid manufactured under the trademark Mexoryl SL by Chimex,
Terephthalylidenedicamphorsulfonic acid manufactured under the trademark Mexoryl SX by Chimex,
Camphor benzalkonium methosulfate manufactured under the trademark Mexoryl SO by Chimex,
Polyacrylamidomethylbenzylidenecamphor manufactured under the trademark Mexoryl SW by Chimex,

Phenylbenzimidazole Derivatives:

Phenylbenzimidazolesulfonic acid marketed in particular under the trademark Eusolex 232 by Merck,
Disodium phenyl dibenzimidazole tetrasulfonate marketed under the trademark Neo Heliopan AP by Haarmann and Reimer,

Anthranilic Derivatives:

Menthyl anthranilate marketed under the trademark Neo Heliopan MA by Haarmann and Reimer,

Imidazoline Derivatives:

Ethylhexyldimethoxybenzylidenedioxoimidazoline propionate,

Benzalmalonate Derivatives:

Polyorganosiloxane containing benzalmalonate functions, for instance Polysilicone-15, marketed under the trademark Parsol SLX by Hoffmann LaRoche,

4,4-Diarylbutadiene Derivatives:

1,1-Dicarboxy(2,2′-dimethylpropyl)-4,4-diphenylbutadiene.

The preferred additional organic photoprotective agents are selected from among:

Ethylhexyl methoxycinnamate,

Homosalate,

Ethylhexyl salicylate,

Octocrylene,

Phenylbenzimidazolesulfonic acid,

Terephthalylidenedicamphorsulfonic acid,

Disodium phenyldibenzimidazoletetrasulfonate,

Benzophenone-3,

Benzophenone-4,

Benzophenone-5,

4-methylbenzylidenecamphor,

Polysilicone-15,

1,1-Dicarboxy(2,2′-dimethylpropyl)-4,4-diphenylbutadiene, and mixtures thereof.

The additional mineral photoprotective agents are selected from among coated or uncoated metal oxide pigments (mean size of the primary particles: generally from 5 nm and 100 nm and preferably from 10 nm and 50 nm), for instance titanium oxide (amorphous or crystallized in rutile and/or anatase form), iron oxide, zinc oxide, zirconium oxide or cerium oxide pigments, which are all UV-photoprotective agents that are well known per se.

The pigments may be coated or uncoated.

The coated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithin is, sodium, potassium, zinc, iron or aluminum salts of fatty acids, metal alkoxides (of titanium or of aluminum), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and consist essentially of a repetition of main units in which the silicon atoms are linked together via oxygen atoms (siloxane bond), optionally substituted hydrocarbon-based radicals being directly attached via a carbon atom to the said silicon atoms.

The term “silicones” also includes the silanes required for their preparation, in particular alkyl silanes.

The silicones used for coating the nanopigments that are suitable for the present invention are preferably selected from the group containing alkyl silanes, polydialkylsiloxanes and polyalkylhydrogenosiloxanes. Even more preferentially, the silicones are selected from the group containing octyltrimethylsilane, polydimethylsiloxanes and polymethylhydrogenosiloxanes.

Needless to say, before being treated with silicones, the metal oxide pigments may have been treated with other surface agents, in particular with cerium oxide, alumina, silica, aluminum compounds or silicon compounds, or mixtures thereof.

The coated pigments are more particularly titanium oxides that have been coated:

with silica, such as the product Sunveil from the company Ikeda and the product Eusolex T-AVO from the company Merck;

with silica and iron oxide, such as the product Sunveil F from the company Ikeda;

with silica and alumina, such as the products Microtitanium Dioxide MT 500 SA and Microtitanium Dioxide MT 100 SA from the company Tayca, Tioveil from the company Tioxide and Mirasun TiW 60 from the company Rhodia;

with alumina, such as the products Tipaque TTO-55 (B) and Tipaque TTO-55 (A) from the company Ishihara and UVT 14/4 from the company Kemira;

with alumina and aluminum stearate, such as the product Microtitanium Dioxide MT 100 TV, MT 100 TX, MT 100 Z and MT-01 from the company Tayca, and the products Solaveil CT-10 W, Solaveil CT 100 and Solaveil CT 200 from the company Uniqema;

with silica, alumina and alginic acid, such as the product MT-100 AQ from the company Tayca;

with alumina and aluminum laurate, such as the product Microtitanium Dioxide MT 100 S from the company Tayca;

with iron oxide and iron stearate, such as the product Microtitanium Dioxide MT 100 F from the company Tayca;

with zinc oxide and zinc stearate, such as the product BR351 from the company Tayca;

with silica and alumina and treated with a silicone, such as the products Microtitanium Dioxide MT 600 SAS, Microtitanium Dioxide MT 500 SAS or Microtitanium Dioxide MT 100 SAS from the company Tayca,

with silica, alumina and aluminum stearate and treated with a silicone, such as the product STT-30-DS from the company Titan Kogyo;

with silica and treated with a silicone, such as the product UV-Titan X 195 from the company Kemira, or the product SMT-100 WRS from the company Tayca;

with alumina and treated with a silicone, such as the products Tipaque TTO-55 (S) from the company Ishihara or UV Titan M 262 from the company Kemira;

with triethanolamine, such as the product STT-65-S from the company Titan Kogyo;

with stearic acid, such as the product Tipaque TTO-55 (C) from the company Ishihara;

with sodium hexametaphosphate, such as the product Microtitanium Dioxide MT 150 W from the company Tayca.

Other titanium oxide pigments treated with a silicone are preferably TiO₂ treated with octyltrimethylsilane and for which the mean size of the elementary particles is from 25 and 40 nm, such as the product marketed under the trademark T 805 by Degussa Silices, TiO₂ treated with a polydimethylsiloxane and for which the mean size of the elementary particles is 21 nm, such as the product marketed under the trademark 70250 Cardre UF TiO2SI3 by Cardre, anatase/rutile TiO₂ treated with a polydimethylhydrogenosiloxane and for which the mean size of the elementary particles is 25 nm, such as the product marketed under the trademark Microtitanium Dioxide USP Grade Hydrophobic by Color Techniques.

The uncoated titanium oxide pigments are marketed, for example, by Tayca under the trademarks Microtitanium Dioxide MT 500 B or Microtitanium Dioxide MT 600 B, by Degussa under the trademark P 25, by Wacker under the trademark Transparent titanium oxide PW, by Myoshi Kasei under the trademark UFTR, by Tomen under the trademark ITS and by Tioxide under the trademark Tioveil AQ.

The uncoated zinc oxide pigments are, for example:

those marketed under the trademark Z-Cote by Sunsmart;

those marketed under the trademark Nanox by Elementis;

those marketed under the trademark Nanogard WCD 2025 by Nanophase Technologies.

The coated zinc oxide pigments are, for example: those marketed under the trademark Z-Cote HP1 by Sunsmart (dimethicone-coated ZnO);

those marketed under the trademark Zinc Oxide CS-5 by Toshibi (ZnO coated with polymethylhydrogenosiloxane);

those marketed under the trademark Nanogard Zinc Oxide FN by Nanophase Technologies (as a 40% dispersion in Finsolv TN, C₁₂-C₁₅ alkyl benzoate);

those marketed under the trademark Daitopersion ZN-30 and Daitopersion ZN-50 by Daito (dispersions in cyclopolymethylsiloxane/oxyethylenated polydimethylsiloxane, containing 30% or 50% of nanozinc oxides coated with silica and polymethylhydrogenosiloxane);

those marketed under the trademark NFD Ultrafine ZnO by Daikin (ZnO coated with perfluoroalkyl phosphate and copolymer based on perfluoroalkylethyl as a dispersion in cydopentasiloxane); those marketed under the trademark SPD-Z1 by Shin-Etsu (ZnO coated with silicone-grafted acrylic polymer, dispersed in cyclodimethylsiloxane);

those marketed under the trademark Escalol Z100 by ISP (alumina-treated ZnO dispersed in an ethylhexyl methoxycinnamate/PVP-hexadecene/methicone copolymer mixture);

those marketed under the trademark Fuji ZnO-SMS-10 by Fuji Pigment (ZnO coated with silica and polymethylsilsesquioxane); those marketed under the trademark Nanox Gel TN by Elementis (ZnO dispersed at a concentration of 55% in C₁₂-C₁₅ alkyl benzoate with hydroxystearic acid polycondensate).

The uncoated cerium oxide pigments are marketed under the trademark Colloidal Cerium Oxide by Rhone-Poulenc.

The uncoated iron oxide nanopigments are marketed, for example, by Arnaud under the trademarks Nanogard WCD 2002 (FE 45B), Nanogard Iron FE 45 BL AQ, Nanogard FE 45R AQ and Nanogard WCD 2006 (FE 45R) or by Mitsubishi under the trademark TY-220,

The coated iron oxide pigments are marketed, for example, by Amaud under the trademarks Nanogard WCD 2008 (FE 45B FN), Nanogard WCD 2009 (FE 45B 556), Nanogard FE 45 BL 345 and Nanogard FE 45 BL or by BASF under the trademark Transparent Iron Oxide.

Mention may also be made of mixtures of metal oxides, especially of titanium dioxide and of cerium dioxide, including the silica-coated equal-weight mixture of titanium dioxide and of cerium dioxide, marketed by Ikeda under the trademark Sunveil A, and also the alumina, silica and silicone-coated mixture of titanium dioxide and of zinc dioxide, such as the product M 261 marketed by Kemira, or the alumina, silica and glycerol-coated mixture of titanium dioxide and of zinc dioxide, such as the product M 211 marketed by Kemira.

The additional photoprotective agents are generally present in the compositions according to the invention in proportions ranging from 0.01% to 20% by weight relative to the total weight of the composition, and preferably ranging from 0.1% to 10% by weight relative to the total weight of the composition.

The compositions according to the invention may also contain agents for artificially tanning and/or browning the skin (self-tanning agents) and more particularly dihydroxyacetone (DHA). They are preferably present in amounts ranging from 0.1% to 10% by weight relative to the total weight of the composition.

The aqueous compositions in accordance with the present invention may also comprise standard cosmetic adjuvants selected especially from among fatty substances, organic solvents, ionic or nonionic, hydrophilic or lipophilic thickeners, softeners, humectants, opacifiers, stabilizers, emollients, silicones, antifoams, fragrances, preservatives, anionic, cationic, nonionic, zwitterionic or amphoteric surfactants, active agents, fillers, polymers, propellants, acidifying or basifying agents or any other ingredient usually used in cosmetics and/or dermatology.

The fatty substances may be an oil or a wax other than the apolar waxes as defined above, or mixtures thereof. The term oil means a compound that is liquid at room temperature. The term wax means a compound that is solid or substantially solid at room temperature and whose melting point is generally greater than 35° C.

Oils that are exemplary may be mentioned include mineral oils (paraffin); plant oils (sweet almond oil, macadamia oil, grapeseed oil or jojoba oil); synthetic oils, for instance perhydrosqualene, fatty alcohols, fatty acids or fatty esters (for instance the C₁₂-C₁₅ alkyl benzoate marketed under the trademark Finsolv TN or Witconol TN by Witco, octyl palmitate, isopropyl lanolate and triglycerides, including capric/caprylic acid triglycerides, and dicaprylyl carbonate marketed under the trademark Cetiol CC by Cognis), oxyethylenated or oxypropylenated fatty esters and ethers; silicone oils (cyclomethicone and polydimethylsiloxanes, or PDMS) or fluoro oils, and polyalkylenes.

Waxy compounds that are exemplary include camauba wax, beeswax, hydrogenated castor oil, polyethylene waxes and polymethylene waxes, for instance the product marketed under the trademark Cirebelle 303 by Sasol.

Among the organic solvents that are exemplary are lower alcohols and polyols. These polyols may be selected from glycols and glycol ethers, for instance ethylene glycol, propylene glycol, butylene glycol, dipropylene glycol or diethylene glycol.

Hydrophilic thickeners that are exemplary include carboxyvinyl polymers such as the Carbopol products (carbomers) and the Pemulen products (acrylate/C₁₀-C₃₀-alkylacrylate copolymer); polyacrylamides, for instance the crosslinked copolymers marketed under the trademarks Sepigel 305 (CTFA name: polyacrylamide/C₁₃₋₁₄ isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulfonic acid polymers and copolymers, which are optionally crosslinked and/or neutralized, for instance the poly(2-acrylamido-2-methylpropanesulfonic acid) marketed by Hoechst under the trademark Hostacerin AMPS (CTFA name: ammonium polyacryloyldimethyltaurate) or Simulgel 800 marketed by SEPPIC (CTFA name: sodium polyacryloyldimethyltaurate/polysorbate 80/sorbitan oleate); copolymers of 2-acrylamido-2-methylpropanesulfonic acid and of hydroxyethyl acrylate, for instance Simulgel NS and Sepinov EMT 10 marketed by SEPPIC; cellulose-based derivatives such as hydroxyethylcellulose; polysaccharides and especially gums such as xanthan gum; and mixtures thereof.

Lipophilic thickeners that are exemplary include synthetic polymers such as poly(C₁₀-C₃₀ alkyl acrylates) marketed under the trademark Intelimer IPA 13-1 and Intelimer IPA 13-6 by Landec, or modified clays such as hectorite and its derivatives, for instance the products marketed under the trademark bentone.

Among the active agents that are exemplary are:

vitamins (A, C, E, K, PP, etc.) and derivatives or precursors thereof, alone or as mixtures;

antipollution agents and/or free-radical scavengers;

depigmenting agents and/or propigmenting agents;

anti-glycation agents;

calmatives;

NO-synthase inhibitors;

agents for stimulating the synthesis of dermal or epidermal macromolecules and/or for preventing their degradation;

agents for stimulating fibroblast proliferation;

agents for stimulating keratinocyte proliferation;

muscle relaxants;

tensioning agents;

matting agents;

keratolytic agents;

desquamating agents;

moisturizers;

anti-inflammatory agents;

agents acting on the energy metabolism of cells;

insect repellants;

substance P or CGRP antagonists;

hair-loss counteractants and/or hair restorers;

anti-wrinkle agents.

Needless to say, one skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be formulated according to techniques that are well known to those skilled in the art. They may be in particular in the form of a simple or complex emulsion (O/W, W/O, O/W/O or W/O/W emulsion) such as a cream, a milk or a cream-gel; in the form of an aqueous gel; in the form of a lotion. They may optionally be packaged as an aerosol and may be in the form of a mousse or a spray.

The compositions according to the invention are preferably in the form of an oil-in-water or water-in-oil emulsion.

The emulsions generally contain at least one emulsifier selected from among amphoteric, anionic, cationic and nonionic emulsifiers, which are used alone or as a mixture. The emulsifiers are appropriately selected according to the emulsion to be obtained (W/O or O/W). The emulsions may also contain stabilizers of other types, for instance fillers, gelling polymers or thickeners.

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of cyclomethicone and of dimethicone copolyol, marketed under the trademark DC 5225 C by Dow Corning, and alkyldimethicone copolyols such as laurylmethicone copolyol marketed under the trademark Dow Corning 5200 Formulation Aid by Dow Corning; cetyldimethicone copolyol, such as the product marketed under the trademark Abil EM 90R by Goldschmidt, and the mixture of cetyldimethicone copolyol, of polyglyceryl isostearate (4 mol) and of hexyl laurate, marketed under the trademark Abil WE 09 by Goldschmidt. One or more co-emulsifiers may also be added thereto, which may be selected advantageously from the group comprising polyol alkyl esters.

Polyol alkyl esters that are especially exemplary include polyethylene glycol esters, for instance PEG-30 dipolyhydroxystearate, such as the product marketed under the trademark Ariacel P135 by ICI.

Glycerol and/or sorbitan esters that are especially exemplary include, for example, polyglyceryl isostearate, such as the product marketed under the trademark Isolan GI 34 by Goldschmidt, sorbitan isostearate, such as the product marketed under the trademark Aracel 987 by ICI, sorbitan glyceryl isostearate, such as the product marketed under the trademark Aracel 986 by ICI, and mixtures thereof.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters, for instance the mixture PEG-100 stearate/glyceryl stearate marketed, for example, by ICI under the trademark Aracel 165; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside marketed, for example, by Henkel under the respective trademarks Plantaren 2000 and Plantaren 1200, cetostearyl glucoside optionally as a mixture with cetostearyl alcohol, marketed, for example, under the trademark Montanov 68 by SEPPIC, under the trademark Tegocare CG90 by Goldschmidt and under the trademark Emulgade KE3302 by Henkel, and also arachidyl glucoside, for example in the form of a mixture of arachidyl alcohol, behenyl alcohol and arachidyl glucoside, marketed under the trademark Montanov 202 by SEPPIC. According to one particular embodiment of the invention, the mixture of the alkylpolyglucoside as defined above with the corresponding fatty alcohol may be in the form of a self-emulsifying composition as described, for example, in WO-A-92/06778.

Among the other emulsion stabilizers that will be used more particularly are isophthalic acid or sulfoisophthalic acid polymers, and in particular phthalate/sulfoisophthalate/glycol copolymers, for example the diethylene glycolphthalate/isophthalate/1,4-cyclohexanedimethanol copolymer (INCI name: Polyester-5) marketed under the trademark Eastman AQ Polymer (AQ35S, AQ38S, AQ55S and AQ48 Ultra) by Eastman Chemical.

When it is an emulsion, the aqueous phase of this emulsion may comprise a nonionic vesicular dispersion prepared according to known processes (Bangham, Standish and Watkins, J. Mol. Biol., 13, 238 (1965), FR-2,315,991 and FR-2,416,008).

The compositions according to the invention are useful in a large number of treatments, whether regime or regimen, especially cosmetic/dermatological treatments, of the skin, the lips and the hair, including the scalp, especially for protecting and/or caring for the skin, the lips and/or the hair, and/or for making up the skin and/or the lips.

The present invention also features the use of the subject compositions as defined above for the manufacture of cosmetic products for treating the skin, the lips, the nails, the hair, the eyelashes, the eyebrows and/or the scalp, especially care products, anti-sun/sunscreen products and makeup products.

The cosmetic compositions according to the invention may be used, for example, as makeup products.

The cosmetic compositions according to the invention may be used, for example, as care products and/or anti-sun protection products for the face and/or the body, of liquid to semi-liquid consistency, such as milks, more or less rich creams, cream-gels and pastes. They may optionally be conditioned in aerosol form and may be in the form of a mousse or a spray.

The compositions according to the invention in the form of vaporizable fluid lotions in accordance with the invention are applied to the skin or the hair in the form of fine particles by means of pressurization devices. The devices in accordance with the invention are well known to those skilled in the art and comprise non-aerosol pumps or “atomizers”, aerosol containers comprising a propellant and also aerosol pumps using compressed air as propellant. These devices are described in U.S. Pat. Nos. 4,077,441 and 4,850,517.

The compositions in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES OF SYNTHESIS

### Example 1: Preparation of 2,4-bis(ethyl 4′-diylaminobenzoate)-6-{[1,3,3,3-tetramethyl-1-[(trimethylsilyl)oxy]disiloxanyl]propyl-3-ylamino}-s-triazine

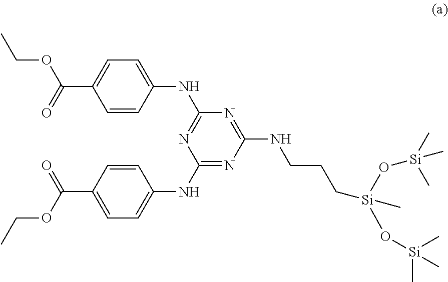 [SMILES: CCOC(=O)c1ccc(Nc2nc(NCCC[Si](C)(O[Si](C)(C)C)O[Si](C)(C)C)nc(Nc3ccc(C(=O)OCC)cc3)n2)cc1]

### First Step: Preparation of 2,4-dichloro-6-{[1,3,3,3-tetramethyl-1-[(trimethylsilyl)oxy]disiloxanyl]propyl-3-ylamino}-s-triazine

1-Amino-3-[1,3,3,3-tetramethyl-1-[(trimethylsilyl)oxy]disiloxanyl]propane (41.7 g, 0.149 mol) and a solution of sodium bicarbonate (11.4 g, 0.135 mol) in 120 ml of water are added dropwise at 0° C. to a solution of cyanuric chloride (25 g, 0.135 mol) in 250 ml of acetone, such that the pH is from 3 and 6.5. At the end of the addition, the pH is 6.5. The mixture is then stirred for 1 hour 30 minutes at 10° C. and then left at the temperature of the laboratory. The precipitate formed is filtered off, washed with water, suction-drained and dried. 55.2 g (yield: 95%) of the expected derivative are obtained in the form of a white powder (m.p.: 59° C.).

### Second Step: Preparation of the Derivative of Example 1

A mixture of the above product (2.1 g, 0.005 mol) and of ethyl para-aminobenzoate (1.65 g, 0.01 mol) suspended in 20 ml of toluene is refluxed for 1 hour 30 minutes. The resulting mixture is cooled and hot heptane is added to the resin obtained. After triturating, filtering and drying, 2.3 g (yield: 67%) of the derivative of Example 1 are obtained in the form of a white powder:

m.p.: 106-108° C.,

UV (ethanol): λmax=311 nm, E1%=1147.

### Example 2: Preparation of 2,4-bis(n-butyl 4′-diylaminobenzoate)-6-{[1,3,3,3-tetramethyl-1-[(trimethylsilyl)oxy]disiloxanyl]propyl-3-ylamino}-s-triazine

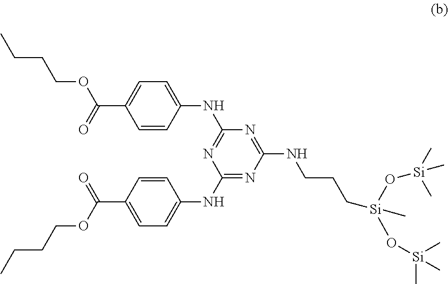 [SMILES: CCCCOC(=O)c1ccc(Nc2nc(NCCC[Si](C)(O[Si](C)(C)C)O[Si](C)(C)C)nc(Nc3ccc(C(=O)OCCCC)cc3)n2)cc1]

A mixture of the product from the first step of Example 1 (16.74 g, 0.0391 mol), n-butyl para-aminobenzoate (15 g, 0.0776 mol) and potassium carbonate (5.36 g, 0.0388 mol) is suspended in 170 ml of toluene and is refluxed for 1 hour 20 minutes, under a nitrogen sparge. The reaction mixture is cooled and 150 ml of dichloromethane are added. The mineral products are filtered off. The filtrate is washed with aqueous bicarbonate solution and then twice with water. After drying the organic phase and evaporating off the solvents, a white powder is obtained. After recrystallization from a 1/15 EtOAc/heptane mixture, 20.1 g (yield: 69%) of the derivative of Example 2 are obtained in the form of a white powder.

m.p.: 111-113° C.,

UV (ethanol): λmax=312 nm, E1%=1055.

### Example 3: Preparation of 2,4-bis(n-pentyl 4′-diylaminobenzoate)-6-{[1,3,3,3-tetramethyl-1-[(trimethylsilyl)oxy]disiloxanyl]propyl-3-ylamino)-s-triazine

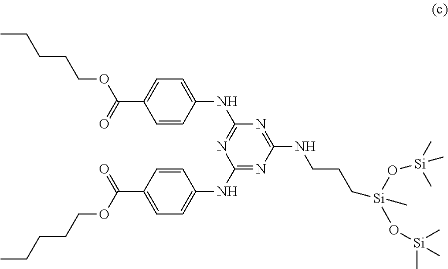 [SMILES: CCCCCOC(=O)c1ccc(Nc2nc(NCCC[Si](C)(O[Si](C)(C)C)O[Si](C)(C)C)nc(Nc3ccc(C(=O)OCCCCC)cc3)n2)cc1]

A mixture of the product from the first step of Example 1 (1 g, 2.3×10⁻³ mol), n-pentyl para-aminobenzoate (0.97 g, 4.6×10⁻³ mol) and sodium bicarbonate (0.39 g, 4.6×10⁻³ mol) in 15 ml of toluene is heated for 20 minutes at a temperature of 115° C. and at a power of 150 watts in a CEM Discover microwave reactor. Dichloromethane is added and the reaction mixture is washed with saturated sodium chloride solution and then twice with water. After drying the organic phase and evaporating off the solvents, a transparent oil is obtained. After purification on a column of silica (eluent: 85/15 heptane/EtOAc), the clean fractions of the derivative of Example 3 (0.9 g, yield: 50%) are obtained in the form of a white powder:

UV (ethanol): λmax=312 nm, E1%=1008.

### Example 4: Preparation of 2,4-bis[(1,1,3,3-tetramethylbutyl)-4′-diylaminobenzamide]-6-{[1,3,3,3-tetramethyl-1-[(trimethylsilyl)oxy]disiloxanyl]propyl-3-ylamino}-s-triazine

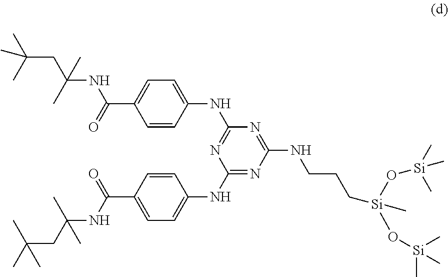 [SMILES: CC(C)(C)CC(C)(C)NC(=O)c1ccc(Nc2nc(NCCC[Si](C)(O[Si](C)(C)C)O[Si](C)(C)C)nc(Nc3ccc(C(=O)NC(C)(C)CC(C)(C)C)cc3)n2)cc1]

### First Step: Preparation of 4-nitro-N-(tert-octyl)benzamide

tert-Octylamine (51.7 g, 0.4 mol) and triethylamine (61.2 ml, 0.44 mol) are introduced into 260 ml of dichloroethane in a reactor. The mixture is heated to 70° C., and 4-nitrobenzoyl chloride (77.9 g, 0.42 mol) is then added portionwise over 50 minutes. The mixture is refluxed for 4 hours. The reaction mixture is poured into ice-water; the resulting mixture is extracted with dichloromethane and dried, and the solvent is evaporated off. The beige-colored precipitate obtained is recrystallized from a mixture of isopropyl ether and ethanol (ratio: 10/1). After drying under vacuum, 84.6 g (yield: 76%) of 4-nitro-N-(tert-octyl)benzamide are obtained in the form of an off-white powder and used without further purification in the following step.

### Second Step: Preparation of 4-amino-N-tert-octyl)benzamide

4-Nitro-N-(tert-octyl)benzamide (30 g, 0.108 mol) dissolved in 200 ml of ethyl acetate in a 500 ml hydrogenator is hydrogenated in the presence of 4.8 g of 10% palladium-on-charcoal containing 50% water, as catalyst (hydrogen pressure: 8-10 bar), at a temperature of 70-75° C. for 1 hour 15 minutes. After filtering, concentrating the solvent and drying under vacuum, 20.4 g (yield: 76%) of 4-amino-N-(tert-octyl)benzamide are obtained in the form of a pale yellow powder and used without further purification in the following step.

### Third Step: Preparation of the Derivative of Example 4

A mixture of the product from the first step of Example 1 (1 g, 2.3×10⁻³ mol), of the product from the preceding step (1.16 g, 4.6×10⁻³ mol) and of sodium bicarbonate (0.39 g, 4.6×10⁻³ mol) in 10 ml of dry toluene is heated for 20 minutes at a temperature of 115° C. and at a power of 150 watts in a CEM Discover microwave reactor. Dichloromethane is added and the reaction mixture is washed with saturated sodium chloride solution and then twice with water. After drying the organic phase and evaporating off the solvents, a pale yellow oil is obtained. After purification on a column of silica (eluent: 70/30 heptane/EtOAc), the clean fractions of the derivative of Example 3 (0.9 g, yield: 45%) are recovered in the form of white flakes:

UV (ethanol): λmax=302 nm, E1%=775.

### Example 6: Preparation of 2,4-bis(methyltrimethylsilyl 4′-diylaminobenzoate)-6-{[1,3,3,3-tetramethyl-1-[(trimethylsilyl)oxy]disiloxanyl]propyl-3-ylamino}-s-triazine

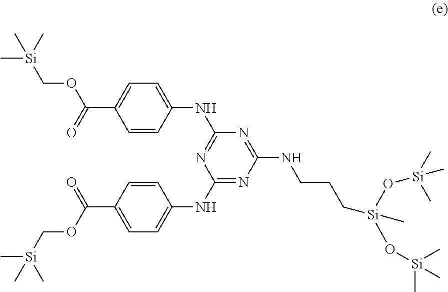

### First Step: Preparation of methyltrimethylsilyl 4-aminobenzoate

Chloromethyltrimethylsilane (38.5 g, 0.314 mol) is added dropwise at 80° C. to a heterogeneous mixture of the potassium salt of para-aminobenzoic acid (50 g, 0.285 mol) in 350 ml of DMF in a reactor. The mixture is refluxed for 3 hours. After cooling, the salts are filtered off and the DMF is evaporated off. The residue is taken up in dichloromethane and dried, and the solvent is evaporated off. The oil obtained is purified by distillation. The fractions that distil at 189° C. under a vacuum of 0.6 mbar are recovered. The oil crystallizes. 50.4 g (yield: 79%) of the derivative of Example 5 are obtained in the form of a white powder and used without further purification in the following step.

### Second Step: Preparation of the Derivative of Example 5

A mixture of the product from the first step of Example 1 (2.1 g, 4.9×10⁻³ mol) and of the derivative from the preceding step (2.19 g, 9.8×10⁻³ mol) in 40 ml of toluene is refluxed for 5 hours while sparging with nitrogen. The mixture is cooled and the solvent is evaporated off. The residue is taken up in dichloromethane and dried, and the solvent is evaporated off. 3 g (yield: 76%) of the derivative of Example 5 are obtained in the form of a pale yellow gum:

UV (ethanol): λmax=311 nm, E1%=907.

### Example 6: Preparation of 2,4-bis(2-ethylhexyl 2′-hydroxy-4′-diylaminobenzoate)-6-{[1,3,3,3-tetramethyl-1-[(trimethylsilyl)oxy]disiloxanyl]propyl-3-ylamino}-s-triazine

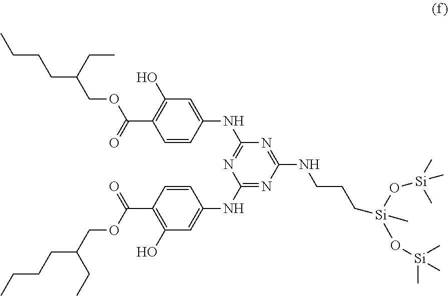 [SMILES: CCCCC(CC)COC(=O)c1ccc(Nc2nc(NCCC[Si](C)(O[Si](C)(C)C)O[Si](C)(C)C)nc(Nc3ccc(C(=O)OCC(CC)CCCC)c(O)c3)n2)cc1O]

A mixture of 2-ethylhexyl 2-hydroxy-4-aminobenzoate (1.4 g, 5.57×10⁻³ mol) and of the product from the first step of Example 1 (1.19 g, 2.78×10⁻³ mol) in 10 ml of toluene is refluxed for 5 hours under a nitrogen sparge. The mixture is cooled and the solvent is evaporated off. The residue is chromatographed on a column of silica (eluent: 9/1 heptane/EtOAc). 1.58 g (yield: 64%) of clean fractions of the derivative of Example 6 are obtained in the form of a white paste:

UV (ethanol): λ=300 nm, E1%=480

λmax=325 nm, E1%=709.

### Example 7: Preparation of the Statistical Derivative of Formula (Ia, III): R₁=n-Butyl, X═O, n=0, B=A, W═H, Z═CH₂, R₇═CH₃, s=0, r=8.1

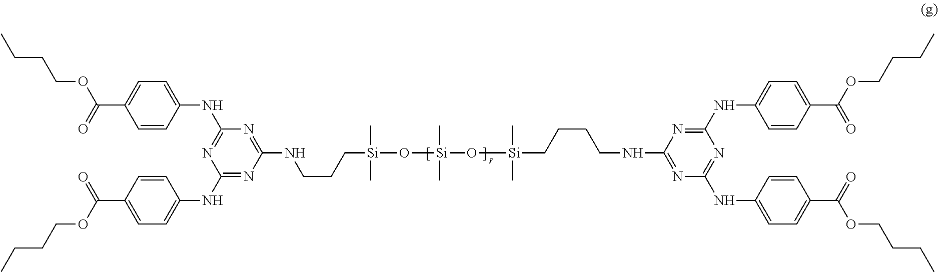

### First Step: Preparation of 2,4-bis(n-butyl 4′-diylaminobenzoate)-6-chloro-s-triazine

n-Butyl para-aminobenzoate (113.94 g, 0.59 mol) and a solution of potassium carbonate (40.68 g, 0.295 mol) in 50 ml of water are simultaneously added dropwise at 5° C. to a solution of cyanuric chloride (54.36 g, 0.295 mol) in 500 ml of dioxane and 50 ml of water, such that the pH is from 3 and 6.5. The mixture is maintained at 5° C. for 1 hour 30 minutes. A precipitate forms in the medium, which corresponds to the monosubstituted s-triazine. The mixture is gradually heated to 70° C. and the second equivalent of potassium carbonate (40.68 g, 0.295 mol) in 50 ml of water is added. Stirring is then continued for 5 hours at 70° C. The reaction mixture is cooled and filtered. The precipitate formed is washed with water, suction-drained and dried. After recrystallization from dioxane/water, followed by drying under vacuum, 52.5 g (yield: 36%) of the first recrystallization crop of 2,4-bis(n-butyl 4′-diylaminobenzoate)-6-chloro-s-triazine are obtained in the form of a white powder.

### Second Step: Preparation of the Derivative of Example 7

A mixture of the preceding product (2 g, 4×10⁻³ mol), of aminopropyl-terminated polydimethylsiloxane (DMS-A-11 from Gelest) (2.13 g, 2×10⁻³ mol) and of pyridine (0.32 ml, 4×10⁻³ mol) in 40 ml of toluene is heated at 70° C. for 5 hours, while sparging with nitrogen. The mixture is cooled, dichloromethane is added and the organic phase is washed three times with water. After drying the organic phase and evaporating off the solvents, a brown oil is obtained. After treating with charcoal in hot ethanol and filtering through Celite, 3.3 g (yield: 70%) of the derivative of Example 7 are obtained in the form of a light-brown gum:

UV (ethanol): λmax=311 nm, E1%=916.

### Example 8: Preparation of butyl 4-{[4-([4-(butoxycarbonyl)phenyl]amino}-6-({3-[diethoxy(methyl)silyl]propyl)amino)-1,3,5-triazin-2-yl]amino}benzoate

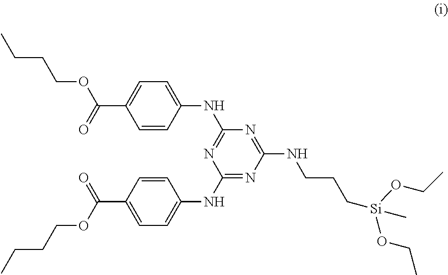 [SMILES: CCCCOC(=O)c1ccc(Nc2nc(NCCC[Si](C)(OCC)OCC)nc(Nc3ccc(C(=O)OCCCC)cc3)n2)cc1]

A heterogeneous mixture of the product from the first step of Example 7 (20 g, 0.04 mol) and of aminopropyldiethoxymethylsilane (15.37 g, 0.08 mol) is gradually heated to 70° C., while sparging with nitrogen. After one hour, the mixture is cooled, dichloromethane is added and the organic phase is washed three times with water. After drying the organic phase and evaporating off the solvents, followed by recrystallization from heptane, 21 g (80% yield) of a white solid of the derivative of Example 8 are obtained:

UV (ethanol): λmax=311 nm, E1%=1197.

### Example 9: Preparation of the Statistical Derivative of Formula (Ia, III) Obtained by Polymerization of the Derivative of Example (8) with D5+MM: R₁=n-Butyl, X═O, n=0, W═H, a=1, b=2, R═CH₃, Z═CH₂

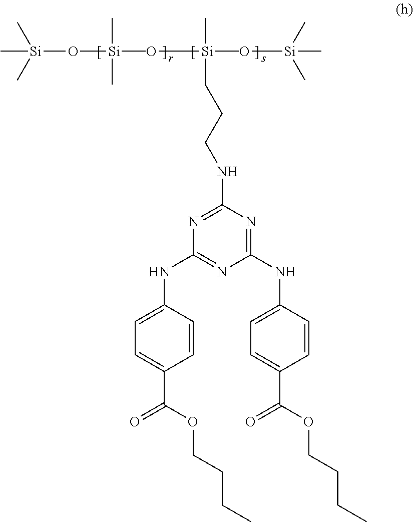

A heterogeneous mixture of the product of Example 8 (1 g, 1.53×10⁻³ mol), decamethylcyclopentasiloxane (D5) (0.57 g, 1.53×10⁻³ mol), hexamethyldisiloxane (MM) (0.062 g, 0.38×10⁻³ mol) and concentrated hydrochloric acid (0.1 ml) is vigorously stirred in a mixture of 10 ml of toluene and 1 ml of water, while sparging with nitrogen. The mixture is gradually heated to 70° C. and left at this temperature for 2 hours. After cooling to room temperature and diluting with water, the mixture is filtered. The precipitate obtained is washed with water and dried. 0.56 g of a white powder of the derivative of Example 9 is thus obtained:

UV (ethanol): λmax=311 nm, E1%=892.

### Examples 10a and 10b: Preparation of the derivatives: butyl 4-[(4-{[4-(butoxycarbonyl)phenyl]amino}-6-{[3-(1-hydroxy-1,3,3,3-tetramethyldisiloxanyl)propyl]amino}-1,3,5-triazin-2-yl)amino]benzoate and dibutyl 4,4′-([6-({3-[dihydroxy(methyl)silyl]propyl)amino)-1,3,5-triazine-2,4-diyl]diimino}dibenzoate obtained via acid treatment of the derivative of Example (2)

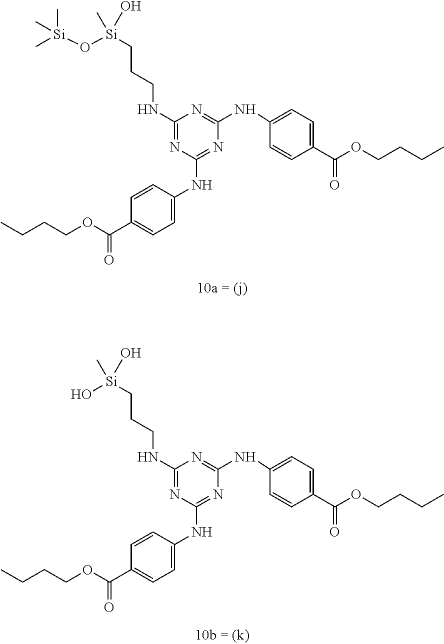

160 ml of 0.1N hydrochloric acid and 340 ml of an 80/20 ethanol/isopropanol mixture are added to the derivative of Example 2 (10 g, 0.013 mol) dissolved in 500 ml of an 80/20 ethanol/isopropanol mixture. The mixture is stirred at the temperature of the laboratory for 5 hours. This solution is neutralized with 0.4% sodium hydroxide to pH 7. One liter of water is added thereto and the solution is freeze-dried. The freeze-dried batches are combined to give 6.5 g of a light-beige powder that contains, in relative percentages by HPLC, about 28% of the derivative of Example 10a and about 10% of the derivative of Example 10b. This powder was fractionated by centrifugal partition chromatography (with two-phase systems composed of heptane, ethyl acetate, methanol and water in different proportions) to give 0.58 g of the derivative of Example 10a in the form of a white powder:

UV (ethanol): λmax=312 nm, E1%=1228 and 0.42 g of the derivative of Example 10b in the form of a white powder.

### Example 11: Preparation of a 7:93 mixture of butyl 4-({4-{[4-(butoxycarbonyl)phenyl]amino}-6-[(2-(1,3,3,3-tetramethyl-1-[(trimethylsilyl)oxy]disiloxanyl)propyl)amino]-1,3,5-triazin-2-yl}amino)benzoate and 2,4-bis(ethyl 4′-diylaminobenzoate)-6-{[1,3,3,3-tetramethyl-1-[(trimethylsilyl)oxy]disiloxanyl]propyl-3-ylamino}-s-triazine

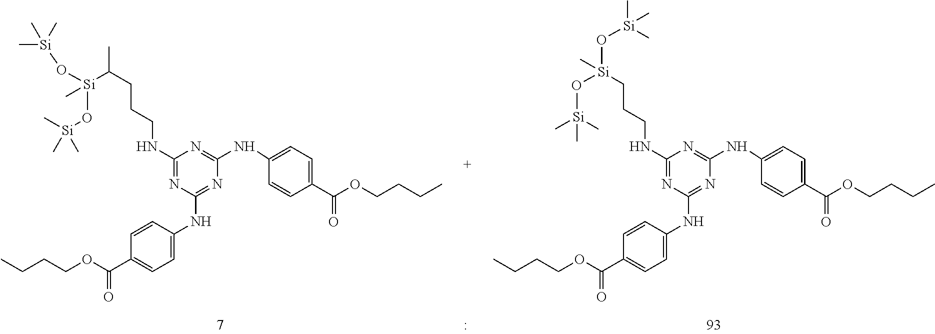 [SMILES: CCCCOC(=O)c1ccc(Nc2nc(NCCCC(C)[Si](C)(O[Si](C)(C)C)O[Si](C)(C)C)nc(Nc3ccc(C(=O)OCCCC)cc3)n2)cc1 ; CCCCOC(=O)c1ccc(Nc2nc(NCCC[Si](C)(O[Si](C)(C)C)O[Si](C)(C)C)nc(Nc3ccc(C(=O)OCCCC)cc3)n2)cc1]

A 7:93 mixture of 2-{1,3,3,3-tetramethyl-1-[(trimethylsilyl)oxy]disiloxanyl}propan-1-amine and of 3-{1,3,3,3-tetramethyl-1-[(trimethylsilyl)oxy]disiloxanyl}propan-1-amine (30 g, 0.11 mol) is added dropwise at 0° C. over 30 minutes to a solution of cyanuric chloride (19.8 g, 0.11 mol) and of lutidine (11.5 g, 0.11 mol) in 100 ml of ethyl acetate. The mixture is stirred at 0° C. for 30 minutes, then at 10° C. for 30 minutes and finally at 20° C. for 30 minutes. Pyridine (17.4 g, 0.22 mol) and n-butyl para-aminobenzoate (41.4 g, 0.22 mol) are then added and the mixture is heated at 70° C. for 3 hours. After cooling the reaction mixture, it is washed with twice 100 ml of saturated sodium chloride solution. This organic phase is passed through a bed of silica and the filter cake is rinsed with 80 ml of ethyl acetate. This organic phase is dried over sodium sulfate and the solvents are evaporated off. The residue obtained is crystallized from heptane. 50.2 g of a pale yellow solid are thus obtained. This solid is recrystallized from a 98/2 heptane/EtOAc mixture to give 49 g (yield: 60%) of a 7:93 mixture of the two isomers of Example 11 in the form of a white powder:

m.p.: 165-167° C.,

UV (ethanol): λmax=312 nm, E1%=1040.

### Example 12: Preparation of a 50:50 mixture of butyl 4-({4-([4-(butoxycarbonyl)phenyl]amino}-6-[(2-{1,3,3,3-tetramethyl-1-[(trimethylsilyl)oxy]disiloxanyl}propyl)amino]-1,3,5-triazin-2-yl}amino)benzoate and 2,4-bis(ethyl 4′-diylaminobenzoate)-6-{[1,3,3,3-tetramethyl-1-[(trimethylsilyl)oxy]disiloxanyl]propyl-3-ylamino}-s-triazine

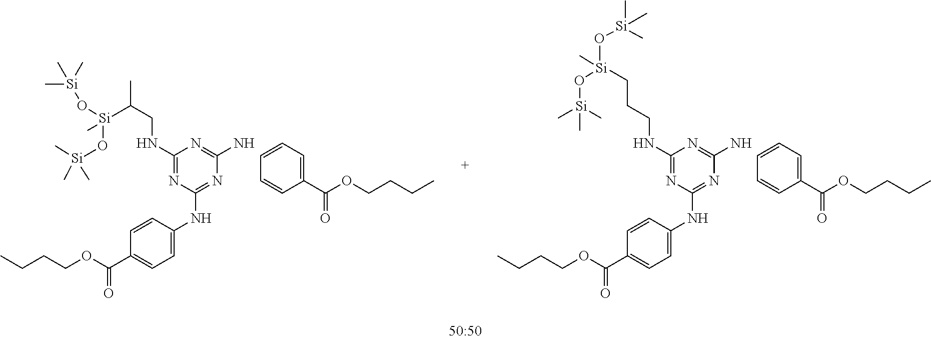

### First Step: Preparation of a 50:60 mixture of 4,6-dichloro-N-(2-(1,3,3,3-tetramethyl-1-[(trimethylsilyl)oxy]disiloxanyl}propyl)-1,3,5-triazin-2-amine and 4,6-dichloro-N-{3-(1,3,3,3-tetramethyl-1-[(trimethylsilyl)oxy]disiloxanyl}propyl)-1,3,5-triazine-2-amine

A 15:85 mixture of 2-{1,3,3,3-tetramethyl-1-[(trimethylsilyl)oxy]disiloxanyl}propan-1-amine and 3-{1,3,3,3-tetramethyl-1-[(trimethylsilyl)oxy]disiloxanyl}propan-1-amine (49.3 g, 0.176 mol) and a solution of sodium bicarbonate (14.8 g, 0.176 mol) in 210 ml of water are added dropwise at 0° C. to a solution of cyanuric chloride (32.5 g, 0.176 mol) in 180 ml of acetone, such that the pH is from 4 and 5.8. At the end of the addition, the pH is 5.3. Stirring is then continued for 1 hour 30 minutes at 10° C. and the mixture is then left at the temperature of the laboratory. The precipitate formed is filtered off, washed with water, suction-drained and dried. 72.4 g (yield: 96%) of the expected isomeric derivatives are obtained in a 15/85 ratio in the form of a white powder (m.p.: 59° C.).

Fractionation of 20 g of the above mixture on two successive silica chromatography columns (eluent: 95/5 heptane/EtOAc) was performed. 3.54 g of a 50/50 isomeric mixture was obtained and used without further purification in the following step.

### Second Step: Preparation of the Derivative of Example 12

A mixture of the above product (3 g, 0.007 mol), of n-butyl para-aminobenzoate (2.7 g, 0.014 mol) and of sodium bicarbonate (1.18 g, 0.014 mol) suspended in 30 ml of toluene is heated at 70° C. for 5 hours. The mixture is cooled and dichloromethane is added. After washing twice with water, the organic phase is dried over sodium sulfate and the solvents are evaporated off. The residue obtained is crystallized from a 50/5 heptane/EtOAc mixture. The precipitate is then purified by column chromatography on silica (eluent: 95/5 CH₂Cl₂/EtOAc) to give 3.7 g (yield: 71%) of a 50/50 mixture of the two isomers of Example 12 in the form of a white powder:

m.p.: 165-167° C.,

UV (ethanol): λmax=312 nm, E1%=1149.

### Example 13: Preparation of the Derivative of Formula (Ia, III): R₁=n-butyl, X═O, n=0, B═CH₃, W═H, Z═CH₂, R₇═CH₃, s=1, r=0

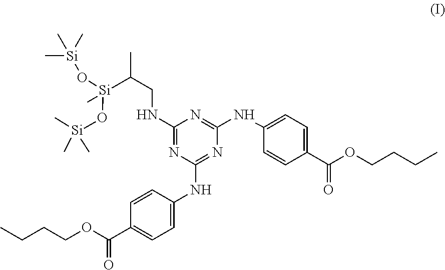 [SMILES: CCCCOC(=O)c1ccc(Nc2nc(NCC(C)[Si](C)(O[Si](C)(C)C)O[Si](C)(C)C)nc(Nc3ccc(C(=O)OCCCC)cc3)n2)cc1]

### First Step: Preparation of 4,6-dichloro-N-(2-{1,3,3,3-tetramethyl-1-[(trimethylsilyl)oxy]disiloxanyl}propyl)-1,3,5-triazin-2-amine

The 15:85 isomeric mixture from the first step of the synthesis of Example 12 was fractionated by centrifugal partition chromatography (two-phase system: 50/49/1 heptane/acetonitrile/water) to give 2.0 g of 4,6-dichloro-N-(2-{1,3,3,3-tetramethyl-1-[(trimethylsilyl)oxy]disiloxanyl}propyl)-1,3,5-triazin-2-amine used as in the following step:

### Second Step: Preparation of the Derivative of Example 13

The above product (2 g, 0.0047 mol) is dissolved in 18 ml of toluene. Pyridine (0.8 ml, 0.009 mol) and n-butyl para-aminobenzoate (1.8 g, 0.009 mol) are added thereto. The mixture is heated at 70° C. with stirring for 3 hours. The solution is cooled and poured onto a bed of silica, and the filter cake is rinsed with 80 ml of toluene. After evaporating off the solvent, the brown-beige solid obtained is crystallized from 30 ml of heptane. 2.2 g (63% yield) of the derivative of Example 13 are thus obtained in the form of a light-beige powder:

m.p.: 149-151° C.,

UV (ethanol): λmax=312 nm, E1%=955.

### Example 14: Preparation of the Derivative of Formula (Ia, III): R₁=n-butyl, X═O, n=0, B═CH₃, W═H, Z═CH₂, R₇═CH₃, s=2, r=0

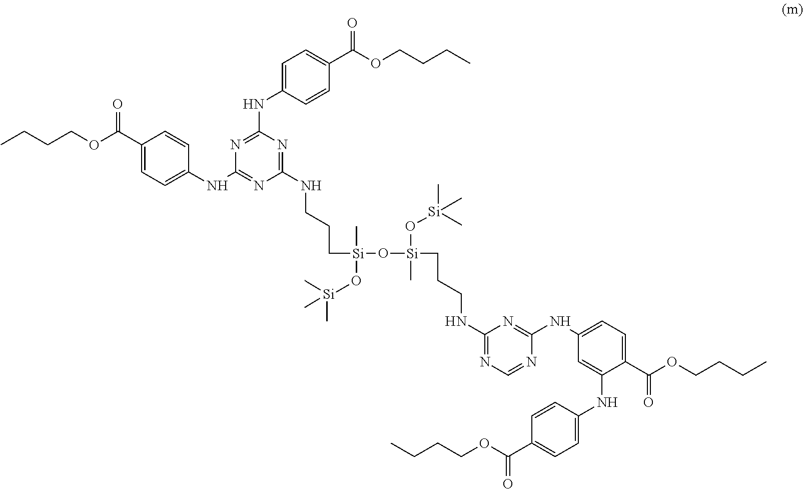 [SMILES: CCCCOC(=O)c1ccc(Nc2nc(NCCC[Si](C)(O[Si](C)(C)C)O[Si](C)(CCCNc3ncnc(Nc4ccc(C(=O)OCCCC)c(Nc5ccc(C(=O)OCCCC)cc5)c4)n3)O[Si](C)(C)C)nc(Nc3ccc(C(=O)OCCCC)cc3)n2)cc1]

25 ml of 1N hydrochloric acid are added to the derivative of Example 2 (1 g, 0.0013 mol) dissolved in 50 ml of an 80/20 ethanol/isopropanol mixture. The mixture is stirred at the temperature of the laboratory for 4 hours. This solution is neutralized with 35% sodium hydroxide to pH 7. The solvents are evaporated off under vacuum. 0.8 g of a light-beige powder is obtained, which contains, as a relative percentage by HPLC, about 37% of the derivative of Example 14. This powder was fractionated by centrifugal partition chromatography (with a two-phase system composed of heptane, ethyl acetate, methanol and water) to give 0.18 g of the derivative of Example 14 in the form of a white powder:

UV (ethanol): λmax=312 nm, E1%=1109.

### Formulation Examples 15 to 17

| | | | |
|---|---|---|---|
| Chemical name | Ex. 15 | Ex. 16 | Ex. 17 |

Phase A

| | | | |
|---|---|---|---|
| s-Triazine compound of Example 13 | 3 | | |
| s-Triazine compound of Example 2 | | 3 | |
| s-Triazine compound of Example 3 | | | 5 |
| Butylmethoxydibenzoylmethane | 2 | 2 | 2 |
| (Parsol 1789) | | | |
| 2,4,6-Tris[p-(2′-ethylhexyl-1′- | 3 | 2 | 3 |
| oxycarbonyl)anilino]-1,3,5-triazine | | | |
| (Uvinul T150) | | | |
| C12-15 alkyl benzoate | 15 | 15 | 15 |
| Cetyl alcohol | 0.5 | 0.5 | 0.5 |
| Stearic acid | 1.5 | 1.5 | 1.5 |
| Glyceryl monostearate/PEG stearate | 1 | 1 | 1 |
| mixture (100 EO) | | | |
| Mixture of cetylstearyl glucoside and | 2 | 2 | 2 |
| of cetyl and stearyl alcohols | | | |
| Dimethicone | 0.50 | 0.50 | 0.50 |
| Triethanolamine | 0.45 | 0.45 | 0.45 |
| Preservative | 1 | 1 | 1 |
| Titanium dioxide | | 5 | |

Phase B

| | | | |
|---|---|---|---|
| Glycerol | 5 | 5 | 5 |
| Complexing agent | 0.1 | 0.1 | 0.1 |
| Monocetyl phosphate | 1 | 1 | 1 |
| Water | qs 100 g | qs 100 g | qs 100 g |

Phase C

| | | | |
|---|---|---|---|
| Xanthan gum | 0.2 | 0.2 | 0.2 |
| Acrylic acid/stearyl methacrylate | 0.2 | 0.2 | 0.2 |
| copolymer | | | |
| Isohexadecane | 1 | 1 | 1 |
| Cyclopentasiloxane | | 1 | |

Phase D

| | | | |
|---|---|---|---|
| Triethanolamine | qs pH | qs pH | qs pH |

Procedure:

The fatty phase (A) is heated to 70° C. The aqueous phase (B) is heated in the final beaker. Phase (C) is prepared: dispersion of the powders in the oil. The fatty phase is emulsified in the aqueous phase by stirring with a rotor-stator. Phase (C) is introduced with faster stirring, and the mixture is then stirred slowly until it has returned to room temperature. The resulting mixture is neutralized (D) and then conditioned.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1.-33. (canceled)

34. A topically applicable, UV-A/UV-B photoprotective and photostabilized cosmetic/dermatological sunscreen composition comprising:
(a) an effective amount of at least one dibenzoylmethane UV-A screening agent;
(b) an effective amount of at least one 1,3,5-triazine compound UV-B screening agent that is otherwise photosensitive in the presence of such amount of said dibenzoylmethane compound; and
(c) an amount of at least one siliceous s-triazine compound substituted with two aminobenzoate or aminobenzamide groups that is effective to photostabilize such effective amount of said at least one 1,3,5-triazine compound UV-B screening agent in the presence of such effective amount of said at least one dibenzoylmethane UV-A screening agent, said at least one siliceous s-triazine compound having the formula (I) below, or tautomeric form thereof: 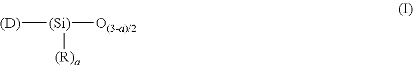
in which:
the radicals R, which may be identical or different, are each a linear or branched and optionally halogenated or unsaturated C₁-C₃₀ alkyl radical, a C₈-C₁₂ aryl radical, a C₁-C₁₀ alkoxy radical or a trimethylsilyloxy group;
a=0 to 3;
the group D is an s-triazine compound of formula (II) below: 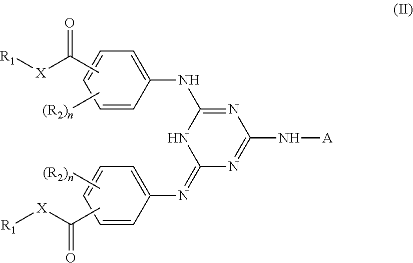
in which:
X is —O— or —NR₃—, wherein R₃ is hydrogen or a C₁-C₅ alkyl radical,
R₁ is a linear or branched and optionally unsaturated C₁-C₂₀ alkyl radical optionally containing a silicon atom, a C₅-C₂₀ cycloalkyl radical optionally substituted with 1 to 3 linear or branched C₁-C₄ alkyl radicals, the group —(CH₂CHR₄—O)ₘR₅ or the group —CH₂—CH(OH)—CH₂—O—R₆,
R₄ is hydrogen or methyl; the group (C═O)XR₁, which may be in an ortho, meta or para position relative to the amino group,
R₅ is hydrogen or a C₁-C₈ alkyl radical,
R₈ is hydrogen or a C₄-C₈ alkyl radical,
m is an integer ranging from 2 to 20,
n=0 to 2,
the radicals R₂, which may be identical or different, are each a hydroxyl radical, a linear or branched C₁-C₈ alkyl radical or a C₁-C₈ alkoxy radical, two adjacent radicals R₂ on the same aromatic nucleus together optionally forming an alkylidenedioxy group in which the alkylidene group contains 1 or 2 carbon atoms,
A is a divalent radical selected from among methylene, —[CHSi(CH₃)₃]—, ethylene and a group corresponding to one of the formulae (III), (IV) and (V) below: 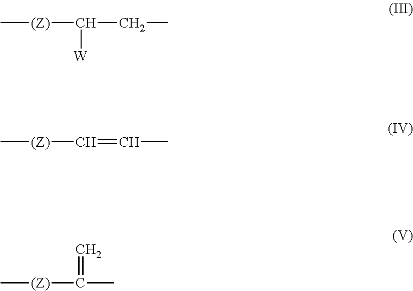
in which:
Z is a linear or branched, saturated or unsaturated C₁-C₁₀ alkylene diradical optionally substituted with a hydroxyl radical or oxygen and optionally containing an amino group, and
W is a hydrogen atom, a hydroxyl radical or a linear or branched, saturated or unsaturated C₁-C₈ alkyl radical, formulated into (e) a topically applicable, cosmetically/dermatologically acceptable support therefor.

35. The cosmetic/dermatological composition as defined by claim 34, in which the compound of formula (I) is in the tautomeric form of formula (I′) below: 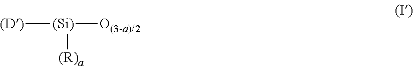
in which the group D′ is an s-triazine compound of formula (II′) below:

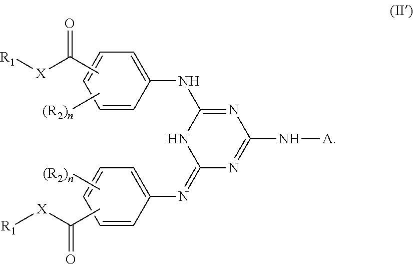

36. The cosmetic/dermatological composition as defined by claim 34, in which the compound of formula (I) also comprises structural units of formula:
(R)_{b}—(Si)(O)_{(4-b)/2}
in which:
R is as defined in formula (I), and
b=1, 2 or 3.

37. The cosmetic/dermatological composition as defined by claim 36, in which the compounds of formula (I) are selected from among those for which at least one of the following characteristics are satisfied:
R is methyl,
a=1 or 2,
X is O,
R₁ is a C₄-C₅ radical,
n=0,
the group (C═O)XR₁ is in the para position relative to the amino group,
Z═—CH₂—, and
W═H.

38. The cosmetic/dermatological composition as defined by claim 34, in which the compounds of formula (I) are selected from among those corresponding to one of the formulae (Ia), (Ib) and (Ic) below: 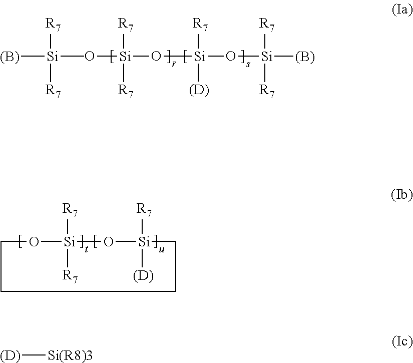
in which:
(D) is as defined in formula (II),
the radicals R₇, which may be identical or different, are each a linear or branched C₁-C₂₀ alkyl, phenyl, 3,3,3-trifluoropropyl or trimethylsilyloxy radical or a hydroxyl radical,
the radicals R₈, which may be identical or different, are each a linear or branched C₁-C₂₀ alkyl or alkenyl radical, or a hydroxyl or phenyl radical, the radicals (B), which may be identical or different, are each a radical R₇ or the radical (D),
r is an integer ranging from 0 to 200 inclusive,
s is an integer ranging from 0 to 50 and if s=0, at least one of the two radicals (B) is (D),
u is an integer ranging from 1 to 10, and
t is an integer ranging from 0 to 10, with the proviso that t+u is greater than or equal to 3, and also the tautomeric forms thereof.

39. The cosmetic/dermatological composition as defined by claim 38, in which the compounds of formula (Ia) or (Ib) are random oligomers or polymers having at least one of the following characteristics:
R₇ is a methyl radical or a hydroxyl radical, and
B is methyl.

40. The cosmetic/dermatological composition as defined by claim 39, in which the compound of formula (I) is selected from among the compounds of formulae (a) to (m) below, and also the tautomeric forms thereof:

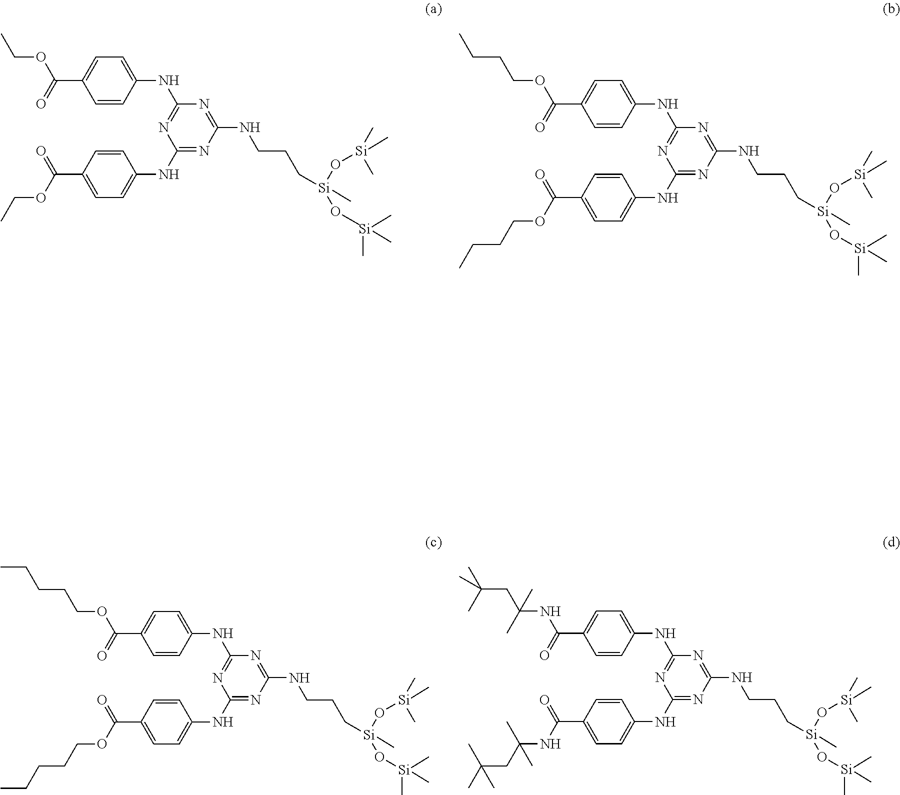

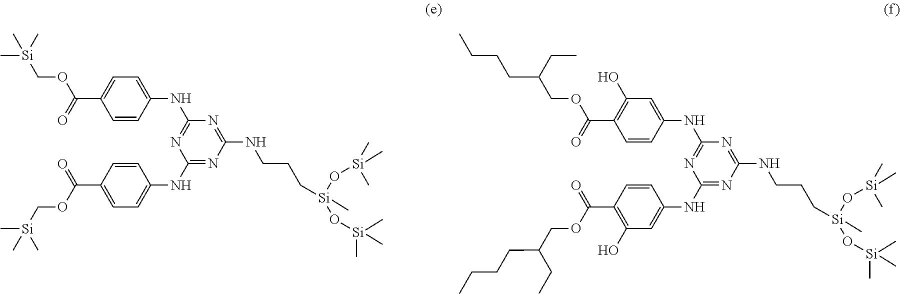

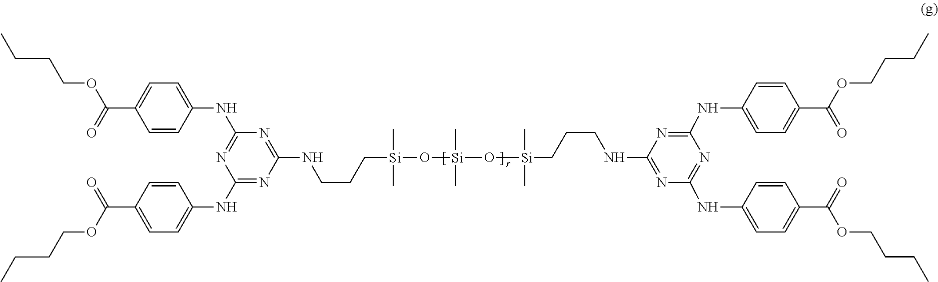

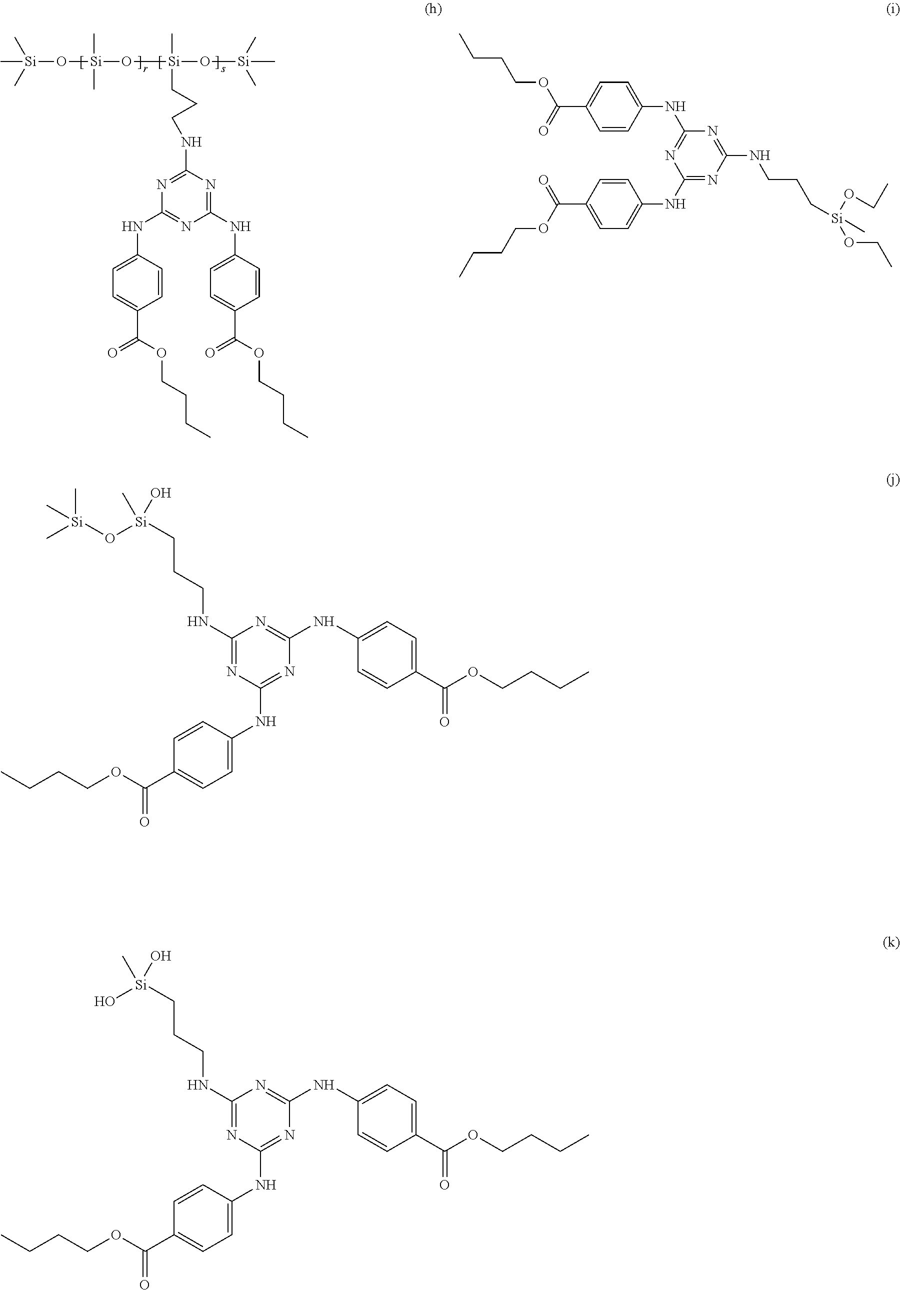

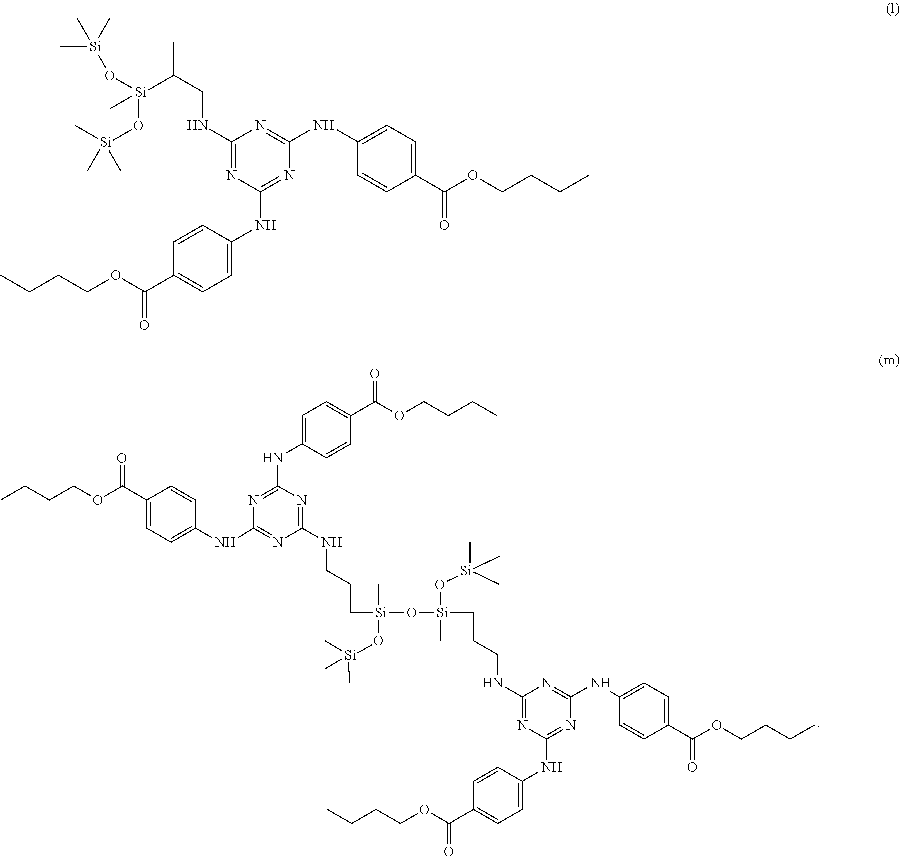

41. The cosmetic/dermatological composition as defined by claim 40, in which the compound of formula (I) is the compound 2,4-bis(n-butyl 4′-diylaminobenzoate)-6-{[1,3,3,3-tetramethyl-1-[(trimethylsilyl)oxy]disiloxanyl]propyl-3-ylamino}-s-triazine of structure (b):

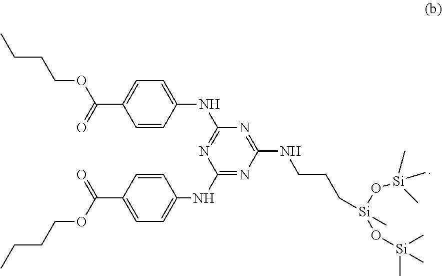 [SMILES: CCCCOC(=O)c1ccc(Nc2nc(NCCC[Si](C)(O[Si](C)(C)C)O[Si](C)(C)C)nc(Nc3ccc(C(=O)OCCCC)cc3)n2)cc1]

42. The cosmetic/dermatological composition as defined by claim 34, in which the s-triazine compound(s) is (are) present in contents ranging from 0.01% to 20% by weight relative to the total weight of the composition.

43. The cosmetic/dermatological composition as defined by claim 34, said at least one dibenzoylmethane compound being selected from among:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane,
2,6-dimethyl-4-tert-butyl-4′-methoxydibenzoylmethane.

44. The cosmetic/dermatological composition as defined by claim 43, said at least one dibenzoylmethane compound comprising 4-(tert-butyl)-4′-methoxydibenzoylmethane.

45. The cosmetic/dermatological composition as defined by claim 34, in which said at least one dibenzoylmethane compound is present in contents ranging from 0.01% to 20% by weight relative to the total weight of the composition.

46. The cosmetic/dermatological composition as defined by claim 34, further comprising at least one non-siliceous lipophilic 1,3,5-triazine compound UV-screening agent selected from among the 1,3,5-triazine compounds of formula (VIII) below: 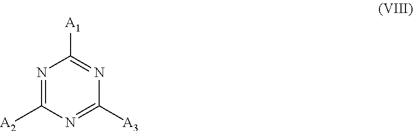
in which the radicals A₁, A₂ and A₃, which may be identical or different, are each a group of formula (IX): 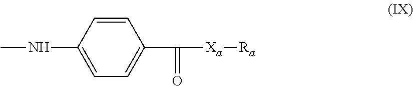
in which:
Xₐ, which may be identical or different, are each oxygen or the radical —NH—;
the radicals Rₐ, which may be identical or different, are each selected from hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a polyoxyethylene radical having from 1 to 6 ethylene oxide units, the terminal OH group of which is methylated; a radical of formula (X), (XI) or (XII) below: 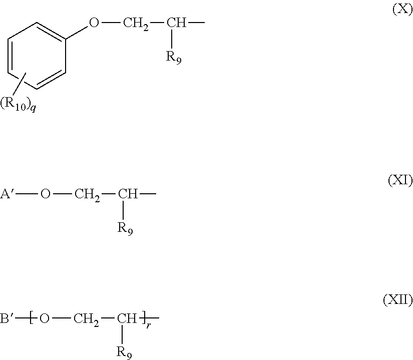
in which:
R₉ is hydrogen or a methyl radical;
R₁₀ is a C₁-C₉ alkyl radical;
q is an integer ranging from 0 to 3;
r is an integer ranging from 1 to 10;
A′ is a C₄-C₈ alkyl radical or a C₅-C₈ cycloalkyl radical; and
B′ is selected from among a linear or branched C₁-C₈ alkyl radical; a C₅-C₈ cycloalkyl radical; an aryl radical optionally substituted with one or more C₁-C₄ alkyl radicals.

47. The cosmetic/dermatological composition as defined by claim 46, in which the 1,3,5-triazine compounds of formula (VIII) are selected from among those in which A₁, A₂ and A₃ are of formula (IX) and have the following characteristics:
one of the radicals Xₐ—Rₐ is a radical —NH—Rₐ wherein Rₐ is selected from among a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical;
the other two radicals Xₐ—Rₐ are a radical —O—Rₐ wherein the radicals Rₐ, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical.

48. The cosmetic/dermatological composition as defined by claim 46, in which the 1,3,5-triazine compounds of formula (VIII) are selected from among those in which A₁, A₂ and A₃ are of formula (IX) and have the following characteristics:
one or two groups Xₐ—Rₐ is a radical —NH—Rₐ, wherein Rₐ is selected from among a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical;
the other or the other two group(s) Xₐ—Rₐ is/are a radical —O—Rₐ wherein the radicals Rₐ, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B′ is a C₁-C₄ alkyl radical and R₁₀ is a methyl radical.

49. The cosmetic/dermatological composition as defined by claim 48, in which said at least one 1,3,5-triazine compound of formula (VIII) comprises 2-[(p-(tert-butylamido)anilino]-4,6-bis[(p-(2′-ethylhexyl-1′-oxycarbonyl)anilino]-1,3,5-triazine corresponding to the following formula: 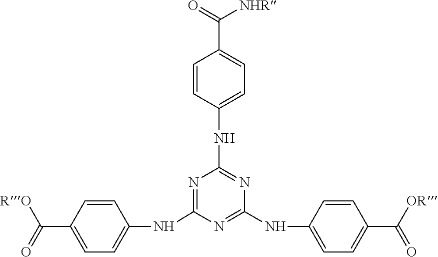
in which R′″ is a 2-ethylhexyl radical and R″ is a tert-butyl radical.

50. The cosmetic/dermatological composition as defined by claim 49, in which said at least one 1,3,5-triazine compound of formula (VIII) is/are selected from among those in which A₁, A₂ and A₃ are of formula (IX) and have the following characteristics:
Xₐ are identical and are oxygen;
the radicals Rₐ, which may be identical or different, are each a C₆-C₁₂ alkyl radical or a polyoxyethylene radical having from 1 to 6 ethylene oxide units and in which the terminal OH group is methylated.

51. The cosmetic/dermatological composition as defined by claim 47, in which said at least one 1,3,5-triazine compound of formula (VIII) comprises 2,4,6-tris[p-(2′-ethylhexyl-1′-oxycarbonyl)anilino]-1,3,5-triazine corresponding to the following formula: 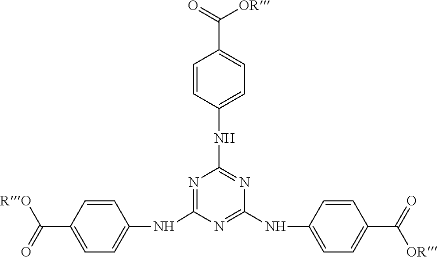
in which R′″ is a 2-ethylhexyl radical.

52. The cosmetic/dermatological composition as defined by claim 34, in which said at least one photosensitive 1,3,5-triazine compound is/are present in contents ranging from 0.01% to 20% by weight relative to the total weight of the composition.

53. The cosmetic/dermatological composition as defined by claim 34, further comprising other organic or inorganic photoprotective agents that are active in the UV-A and/or UV-B range and that are water-soluble or liposoluble or insoluble in the conventional cosmetic solvents.

54. The cosmetic/dermatological composition as defined by claim 53, comprising additional organic photoprotective agents selected from among anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis(benzoazolyl) derivatives; p-aminobenzoic acid (PABA) derivatives; methylene bis(hydroxyphenylbenzotriazole) derivatives; benzoxazole derivatives; screening polymers and screening silicones; α-alkylstyrene-derived dimers; 4,4-diarylbutadienes and mixtures thereof.

55. The cosmetic/dermatological composition as defined by claim 54, comprising additional organic UV screening agent(s) selected from among the following compounds:
Ethylhexyl Methoxycinnamate;
Homosalate;
Ethylhexyl Salicylate;
Octocrylene;
Phenylbenzimidazole Sulfonic Acid;
Benzophenone-3;
Benzophenone-4;
Benzophenone-5;
n-Hexyl 2-(4-diethylamino-2-hydroxybenzoyl)benzoate;
4-Methylbenzylidene camphor;
Terephthalylidene Dicamphor Sulfonic Acid;
Disodium Phenyl Dibenzimidazole Tetrasulfonate;
Methylene bis-Benzotriazolyl Tetramethylbutylphenol;
Ethylhexyl triazone,
Bis-Ethylhexyloxyphenol Methoxyphenyl Triazine;
Diethylhexyl Butamido Triazone;
2,4,6-Tris(dineopentyl 4′-aminobenzalmalonate)-s-triazine;
2,4,6-Tris(diisobutyl 4′-aminobenzalmalonate)-s-triazine;
2,4,6-Tris(biphenyl-4-yl-1,3,5-triazine);
2,4,6-Tris(terphenyl)-1,3,5-triazine;
Drometrizole Trisiloxane;
Polysilicone-15;
Dineopentyl 4′-methoxybenzalmalonate
1,1-Dicarboxy-(2,2′-dimethylpropyl)-4,4-diphenylbutadiene;
2,4-Bis[5-1 (dimethylpropyl)benzoxazol-2-yl-(4-phenyl)-imino]-6-(2-ethylhexyl)-imino-1,3,5-triazine;
and mixtures thereof.

56. The cosmetic/dermatological composition as defined by claim 53, comprising additional inorganic photoprotective agents selected from among treated or untreated metal oxide pigments.

57. The cosmetic/dermatological composition as defined by claim 56, said pigments comprising treated or untreated titanium, zinc, iron, zirconium or cerium oxides and mixtures thereof.

58. The cosmetic/dermatological composition as defined by claim 34, further comprising at least one artificial tanning and/or browning agent for the skin.

59. The cosmetic/dermatological composition as defined by claim 34, further comprising at least one adjuvant selected from among fatty substances, organic solvents, ionic or non-ionic, hydrophilic or lipophilic thickeners, demulcents, humectants, opacifiers, stabilizers, emollients, silicones, anti-foaming agents, fragrances, preservatives, anionic, cationic, non-ionic, zwitterionic or amphoteric surfactants, active agents, fillers, polymers, propellants and basifying or acidifying agents.

60. The cosmetic/dermatological composition as defined by claim 34, formulated as an oil-in-water or water-in-oil emulsion.

61. The cosmetic/dermatological composition as defined by claim 34, formulated as a product for the cosmetic treatment of the skin, lips, nails, hair, eyelashes, eyebrows and/or scalp.

62. The cosmetic/dermatological composition as defined by claim 34, formulated as a care product for the skin, lips, nails, hair and/or scalp.

63. The cosmetic/dermatological composition as defined by claim 34, formulated as a makeup product.

64. A regime or regimen for photoprotecting a keratinous substrate against the damaging effects of UV-A radiation, comprising topically applying thereon a thus effective amount of the cosmetic/dermatological composition as defined by claim 34.

65. A regime or regimen for photoprotecting the skin, hair, lips, scalp, nails, eyelashes and/or eyebrows against the damaging effects of UV-A radiation, comprising topically applying thereon a thus effective amount of the cosmetic/dermatological composition as defined by claim 34.

66. A process for improving the stability to UV radiation of a photosensitive 1,3,5-triazine compound in the presence of a dibenzoylmethane compound, comprising formulating therewith a thus effective amount of at least one siliceous s-triazine compound substituted with two aminobenzoate or aminobenzamide groups, or a tautomeric form thereof.
